(19) United States
(12) Patent Application Publication
(10) Pub. No.: US 2026/0013863 A1
(43) Pub. Date: Jan. 15, 2026

(54) SPIRAL-LIKE DEVICES FOR MODULATING BLOOD FLOW AND RELATED METHODS

(71) Applicant: EDWARDS LIFESCIENCES CORPORATION, Irvine, CA (US)

(72) Inventors: Yevgeniy Vyatcheslavovich Fedotov, Ladera Ranch, CA (US); Afshin Moien Jahromi, Orange, CA (US); Anne Bernadette Aragon Alcasid, Costa Mesa, CA (US); Michael G. Valdez, Riverside, CA (US)

(21) Appl. No.: 19/327,406

(22) Filed: Sep. 12, 2025

Related U.S. Application Data
(63) Continuation of application No. PCT/US2024/019170, filed on Mar. 8, 2024.
(60) Provisional application No. 63/489,846, filed on Mar. 13, 2023.
(60) Provisional application No. 63/604,792, filed on Nov. 30, 2023.

(51) Int. Cl. A61B 17/12; A61B 17/00

(52) U.S. Cl. CPC A61B 17/12036; A61B 17/12109; A61B 17/12145; A61B 2017/00867

(57) ABSTRACT

An implantable device may include a support frame and a coiled wire forming a coil stack, wherein the coiled wire has a first end and a second end, and is adapted to flex between a collapsed configuration and an extended configuration, and wherein the first end is coupled to or formed from the support frame and the second end is extendable and collapsible relative to the support frame. An implantable device may include a jacket surrounding at least a portion of the coiled wire. An implantable device may include a plurality of coiled wires forming a coil stack. An implantable device may include a coilable flow occlusion strip adapted to flex between a collapsed configuration and an extended configuration, and wherein the first end is coupled to the support frame and the second end is extendable and collapsible relative to the support frame.

### CROSS-REFERENCE TO RELATED APPLICATIONS

This application is continuation of International Application No. PCT/US2024/019170, filed Mar. 8, 2024, which claims the benefit of U.S. Provisional Patent Application Ser. No. 63/489,846, filed Mar. 13, 2023, and U.S. Provisional Patent Application Ser. No. 63/604,792, filed Nov. 30, 2023, the contents of each of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical devices and procedures, and more specifically to the field of blood flow management in blood vessels. Described herein are devices and methods for modulating blood flow through a blood vessel.

### BACKGROUND

Chronic kidney disease (CKD) is a common comorbidity with many patients who suffer from chronic Heart Failure (HF). HF patients may also have elevated right atrium pressure, which may impair kidney function. In HF patients with elevated right atrium pressure, the kidneys may attempt to perform a diuresis process, but such a process may be difficult to perform efficiently due to the elevated pressure. For example, elevated right atrium pressure may hinder the ability of the kidneys to drive forward the flow of blood for accomplishing proper and efficient diuresis. Such unbalanced pressure coupled with the typical poor kidney efficiency of CKD patients may lead to an unending cycle of fluid overload for a person, which may result in an increase in congestion and heart failure admissions to the hospital.

Further, heart valve diseases, such as aortic stenosis, mitral regurgitation, or tricuspid regurgitation, can prevent heart valves from closing properly. These conditions can lead to problems like reduced blood flow efficiency, causing the heart to work harder to compensate. Additionally, these disorders may cause increased right atrial pressure. Increased right atrial pressure is transmitted to the central and hepatis veins, leading to hepatosplenomegaly and ascites, which are present in 90% of patients with tricuspid regurgitation. These diseases can result in symptoms such as chest pain, shortness of breath, fatigue, and, if left untreated, can lead to complications like heart failure, arrhythmias, and even damage to the heart muscle.

### SUMMARY

In some aspects, the techniques described herein relate to an implantable device for modulating blood flow through a blood vessel, the implantable device including: a support frame; a coiled wire forming a coil stack, wherein the coiled wire is adapted to flex between a collapsed configuration and an extended configuration, wherein the coiled wire has a first end and a second end, and wherein the first end is coupled to or formed from the support frame and the second end is extendable and collapsible relative to the support frame; and a jacket surrounding at least a portion of the coiled wire.

In some aspects, the techniques described herein relate to an implantable device for modulating blood flow through a blood vessel, the implantable device including: a support frame; and a plurality of coiled wires forming a coil stack, wherein each coiled wire is adapted to flex between a collapsed configuration and an extended configuration, wherein each coiled wire has a fixed end and a terminus, and wherein the fixed end of each coiled wire is coupled to or formed from the support frame and the terminus of each coiled wire is extendable and collapsible relative to the support frame, and wherein each coiled wire includes a respective jacket surrounding at least a portion of each coiled wire.

In some aspects, the techniques described herein relate to an implantable device for modulating blood flow through a blood vessel, the implantable device including: a support frame; and a coilable flow occlusion strip adapted to flex between a collapsed configuration and an extended configuration, wherein the coilable flow occlusion strip has a first end and a second end, and wherein the first end is coupled to the support frame and the second end is extendable and collapsible relative to the support frame.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates an embodiment of a device for modulating blood flow implanted in a blood vessel.

FIG. 1B illustrates an embodiment of a device for modulating blood flow implanted in a blood vessel.

FIG. 1C illustrates an embodiment of a device for modulating blood flow implanted in a blood vessel and electrically coupled to a control device.

FIG. 1D illustrates an embodiment of a device for modulating blood flow implanted in a blood vessel and electrically coupled to a control device.

FIG. 1E illustrates a block diagram of an embodiment of a device for modulating blood flow.

FIG. 2A illustrates a top view of an embodiment of a device for modulating blood flow.

FIG. 2B illustrates a bottom view of the device of FIG. 2A.

FIG. 3A illustrates a side view of one embodiment of a device for modulating blood flow in a collapsed configuration.

FIG. 3B illustrates a side view of one embodiment of a device for modulating blood flow in an extended configuration.

FIG. 3C illustrates a side view of an embodiment of a device for modulating blood flow in an intermediate configuration.

FIG. 4 illustrates a side view of one embodiment of the device in a fully open configuration.

FIG. 5 illustrates a side view of another embodiment of the device in a fully open configuration.

FIG. 6A illustrates a side view of one embodiment of a flow modulating device in an expanded configuration.

FIG. 6B illustrates a bottom view of the embodiment of FIG. 6B in a collapsed configuration.

FIG. 7A illustrates a perspective view of an embodiment of a flow modulating device in a closed configuration.

FIG. 7B illustrates a perspective view of the embodiment of FIG. 7A in a partially open configuration.

FIG. 7C illustrates a perspective view of the embodiment of FIG. 7A in a closed configuration.

FIG. 8A illustrates a schematic of an embodiment of a system for modulating blood flow through a blood vessel.

FIG. 8B illustrates a schematic of an embodiment of a system for modulating blood flow through a blood vessel.

FIG. 9 illustrates an example method for modulating blood flow in a blood vessel.

FIG. 10 illustrates a schematic representation of portions of a subject that may include a flow modulating device implanted therein.

FIG. 11 illustrates a top view of an embodiment of a system for modulating blood flow through a blood vessel.

FIG. 12A illustrates a cross-sectional view of an embodiment of a coiled wire and surrounding jacket of a system for modulating blood flow through a blood vessel along line A-A of the flexible member of FIG. 11.

FIG. 12B illustrates a cross-sectional view of an embodiment of a coiled wire and surrounding jacket of a system for modulating blood flow through a blood vessel along line A-A of the flexible member of FIG. 11.

FIG. 12C illustrates a cross-sectional view of an embodiment of a coiled wire and surrounding jacket of a system for modulating blood flow through a blood vessel along line A-A of the flexible member of FIG. 11.

FIG. 12D illustrates a cross-sectional view of an embodiment of a coiled wire and surrounding jacket of a system for modulating blood flow through a blood vessel.

FIG. 13 illustrates a side-view of a system for modulating blood flow through a blood vessel in a closed configuration.

FIG. 14 illustrates a side-view of a system for modulating blood flow through a blood vessel in an open configuration.

FIG. 15 illustrates a top view of a system for modulating blood flow through a blood vessel in a closed configuration.

FIG. 16 illustrates a side view of a system for modulating blood flow through a blood vessel in an extended configuration.

FIG. 17 illustrates an embodiment of a device for modulating blood flow implanted at least partially within the superior vena cava.

FIG. 18 illustrates an embodiment of a device for modulating blood flow implanted within the superior vena cava.

FIG. 19 illustrates an embodiment of a device for modulating blood flow implanted within the pulmonary artery.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating, regulating, and/or balancing of blood flow through a blood vessel. The modulating, regulating, and/or balancing of blood flow may be performed by the devices described herein to occlude, partially occlude, and/or otherwise manage or regulate blood flow to or through a portion of a blood vessel. In some examples, such modulation, regulating, and/or balancing of blood flow to or through a blood vessel may result in additionally modulating pressure in the right atrium of the heart and/or other vessels or organs of the body. The terms flow modulating system or device, flow regulating system or device, and flow balancing system or device are considered synonymous and can be used interchangeably herein.

The examples presented herein may relate to providing devices, methods, and/or methods of treatment (MOTs) for modulating, regulating and/or otherwise managing blood flow to or through one or more blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, a flow modulation device is synonymous with a pressure regulating device (i.e., a flow regulator is synonymous with a pressure regulator). In some examples, the devices described herein may include blood flow management devices for reducing blood flow through a blood vessel, such as the superior vena cava (SVC), the inferior vena cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or heart failure (HF). For example, patients with CKD and/or HF may exhibit reduced kidney function when pressure in the right atrium of the heart is above a predefined pressure threshold. The predefined pressure threshold may be used as a basis to determine whether a patient is exhibiting low vessel pressure (e.g., below a predefined pressure threshold) or high vessel pressure (e.g., above a predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases or other vessel pressure increases and/or pressure variations. In particular, the devices, methods, and/or MOTs described herein can be used to reduce and maintain low pressure in the right atrium or any pressure arising in any vessel, which provides a technical effect of enabling the kidneys to effectively filter blood in an improved manner over conventional pressure reducing systems and techniques.

In addition, the devices, methods, and/or MOTs described herein can solve a further technical problem of accumulation of blood in the venous system. For example, the devices described herein may be used to reduce the accumulation of blood in the venous system, which can provide an advantage and technical effect of ensuring that pressure is not increased in the SVC and/or the IVC. Such devices can advantageously eliminate excessive hospital readmissions and/or can provide for a long-term blood flow management therapy, improving both quality of life and overall survival rates and with a lower cost to a healthcare system. Furthermore, the devices, methods, and/or MOTs described herein can be used to solve a further technical problem of regulating (e.g., modulating) blood flow return, thus further mitigating pressure build-up in the right atrium or pressure build-up in any bodily vessel. The examples described herein can perform blood flow management actively and/or passively to assist in reducing and/or maintaining right atrium pressures or any vessel pressures to a relatively low pressure even when a surge in blood volume occurs in one or more vessels of the venous system.

Disclosed herein are systems and methods for modulating blood flow through a blood vessel. The systems and devices described herein can involve active and/or passive mechanisms for managing blood flow.

As used herein, the term “active” with respect to blood flow management may represent operations carried out by the devices described herein using power and/or controller induced movement. For example, actively moving a portion of the devices described herein, for example extending or collapsing a spiral valve, may include the use of battery power, wall outlet power, magnetic field induction, electromagnetic field induction, magnetic polarization, a piston-based system, a valve-based system (e.g., with a manifold), hydraulics, pneumatics, optical actuators, thermal actuators, and/or other actuator using electrical or inductive power.

In some implementations, an active control mechanism may include a microcontroller and/or a power source implanted with or integrated with the flow management device. Alternatively, or additionally, an active control mechanism can include a microcontroller and/or a power source in a remote-control device, external to the body, or in an implanted remote device (e.g., subcutaneously, intravascularly, etc.), for example. The remote-control device may be in wireless communication with the implanted device or connected to the implanted device through one or more leads.

In any of the embodiments described herein, an active mechanism may include a linear actuator coupled to a control element of the flow management device. The linear actuator tensions the control element to position the valve of the flow management device in the restricted blood flow state. Alternatively, the linear actuator releases tension in the control element to position the flow occlusion strip or elongate membrane of the spiral valve in the unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote-control device, or otherwise in wireless or electrical communication with a flow management system. The sensor can be a strain gauge, a piezoelectric sensor, a capacitance sensor, a vacuum pressure sensor, or the like, such that the sensor senses a pressure in the blood vessel.

In any of the embodiments described herein, the linear actuator may be an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the flow occlusion strip or elongate membrane of the valve to an unrestricted blood flow state.

In any of the embodiments described herein, the linear actuator is a thermal linear actuator having a piston coupled to the control element. For example, increasing a temperature of a thermal sensitive fluid (e.g., via a heat source, changes in body temperature, etc.) causes the piston to compress the fluid to tension the control element to move the flow occlusion strip or elongate membrane of the valve into the restricted blood flow state. Alternatively, decreasing the temperature of the thermal sensitive fluid causes the piston to decompress the fluid to release tension in the control element to move the flow occlusion strip or elongate membrane of the valve to the unrestricted blood flow state. In further implementations, the linear actuator may include an optical actuator.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, etc.), anatomy responses (e.g., changes in vessel inner diameter, intra-vessel pressure, etc.), blood movement, or the like.

In some examples described herein, a passive mechanism can include a control element, such as a rigid push or pull member (e.g., a rod or rigid wire), a flexible tension member, a flexible wire, a suture, a string, a cable, or the like. One or more control elements can be coupled to a body to be moved (or actuated), such as one or more flow occlusion strips or a portion thereof, elongate membrane, coilable strip, leaflets, cage, flaps, valves, valve portions (e.g., used to restrict blood flow through a blood vessel), paddle(s), or another component. For example, the control element can be coupled to a component that moves in response to an external force (e.g., an increase in blood pressure, or a change in blood flow direction). In some examples, the control element is coupled to a helical tube that expands and contracts with a blood vessel in which it is positioned, and the movement of the helical tube moves the control element. In some examples, a control member can be used to actuate the valve in response to the movement of the frame (or helical tube) due to a change in blood pressure within a blood vessel.

In some examples described herein, a passive mechanism can include a spring, or clastic member (such as a flexible commissure). One or more springs or elastic members can be coupled to a body to be moved (or actuated), such as one or more flow occlusion strips or a portion thereof, elongate membrane, coilable strip, leaflets, cage, flaps, valves, or valve portions (e.g., used to restrict blood flow through a blood vessel), paddle(s), or another component. For example, a spring can be used to bias the flow occlusion strip, elongate membrane, coilable strip, leaflet, cage, flap, valve, paddle, etc. in an initial position (e.g., under relatively lower blood pressure conditions). An externally applied force can cause the flow occlusion strip, elongate membrane, coilable strip, leaflet, cage, flap, valve, paddle, etc. to move and extend the spring (e.g., under relatively higher blood pressure conditions, for example wherein increased blood flow applies pressure against the leaflet, flap, valve, flow occlusion strip, elongate membrane, coilable strip). In the absence of the applied force, the spring can recompress, thereby bringing the leaflet, flap, valve, cage, flow occlusion strip elongate membrane, or coilable strip back to its initial position.

In some examples described herein, a passive mechanism can include more than one mode of operation. For example, a body (or member or component) of a blood flow regulator (or restrictor) described herein can move in a particular way in response to a first externally applied force, and then the body (or member or component) of a blood flow regulator (or restrictor) described herein can move in a different way in response to a second externally applied force. In some cases, the first externally applied force and the second externally applied force are applied from the same external force (e.g., blood pressure), with different quantitative ranges. In some cases, the first externally applied force and the second externally applied force are applied in opposite directions (e.g., different directions of blood flow). For example, a leaflet, flap, valve, cage, paddle, elongate membrane, coilable strip, one or more coiled wires, or flow occlusion strip or portion thereof (e.g., used to restrict blood flow through a blood vessel) can move in a first mode in response to an increase in blood pressure within a first blood pressure range and move in a second mode in response to an increase in blood pressure within a second blood pressure range that is different (e.g., higher) than the first blood pressure range. The first mode can be that an end of the leaflets, cage, flaps, valve, elongate membrane, coilable strip, one or more coiled wires, flow occlusion strip portions, etc. move towards one another to further restrict blood flow within the blood vessel, and the second mode can be that the leaflets, cage, flaps, paddle, valve, elongate membrane, coilable strip, one or more coiled wires, or flow occlusion strip portions prolapse, thereby moving away from one another to increase the blood flow within the blood vessel.

In some examples described herein, a flow modulating device (e.g., a flow restrictor) described herein can include more than one passive mechanism. For example, a flow regulating device can contain a first passive mechanism that can move in a first mode in response to an increase in blood pressure within a first blood pressure range, and a second passive mechanism that can move in a second mode in response to an increase in blood pressure within a second blood pressure range that is different (e.g., higher) than the first blood pressure range. The first passive mechanism can include leaflets, flaps, or valve portions that move towards one another to further restrict blood flow within the blood vessel, and the second passive mechanism can include an inner valve that can move (e.g., axially) to open additional channels through the flow regulator device to increase the blood flow. In some cases, the inner valve can be biased using a passive element such as one or more springs, such that it will return to its initial position after the blood pressure decreases (e.g., back into a first blood pressure range).

Any of the implantable or flow modulating devices, or portion thereof, described herein may comprise or be coated with a polymer (e.g., silicones, poly (urethanes), poly (acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly (ethylene terephthalate), while the nonwoven fabrics may be made from expanded poly (tetrafluoroethylene). Some textiles may also or alternatively include silk or silk-based materials.

Further, any of the pull wires, sutures, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

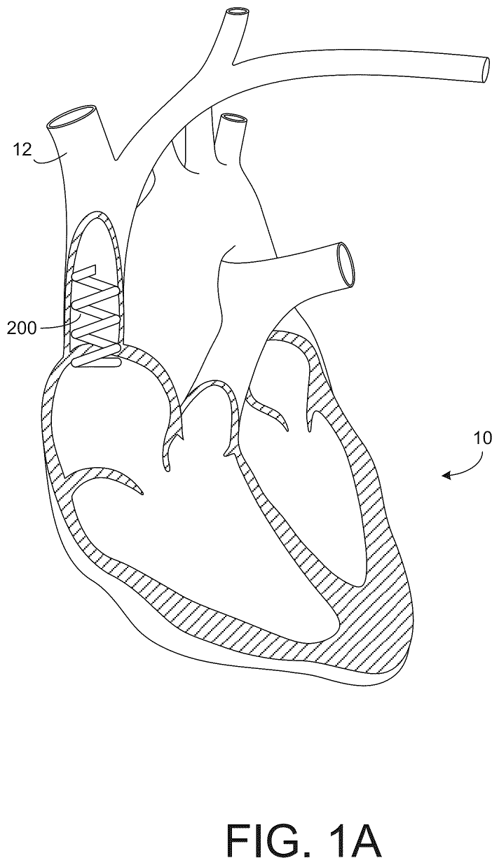

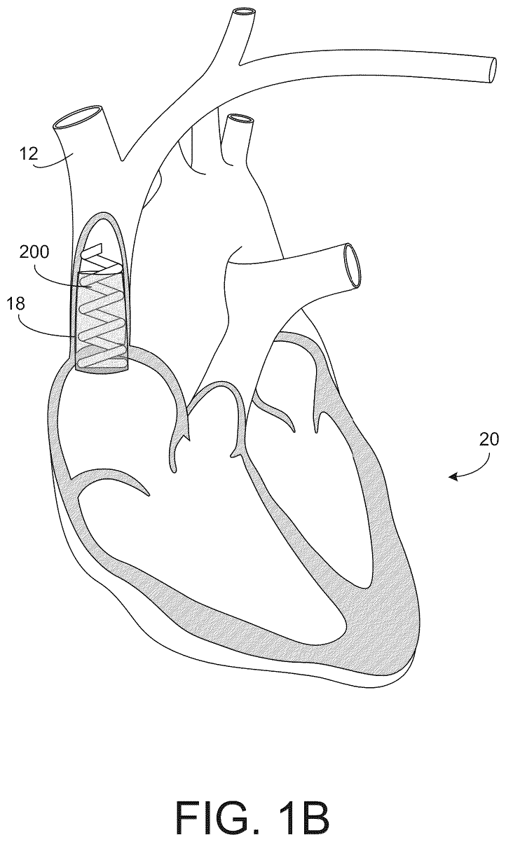

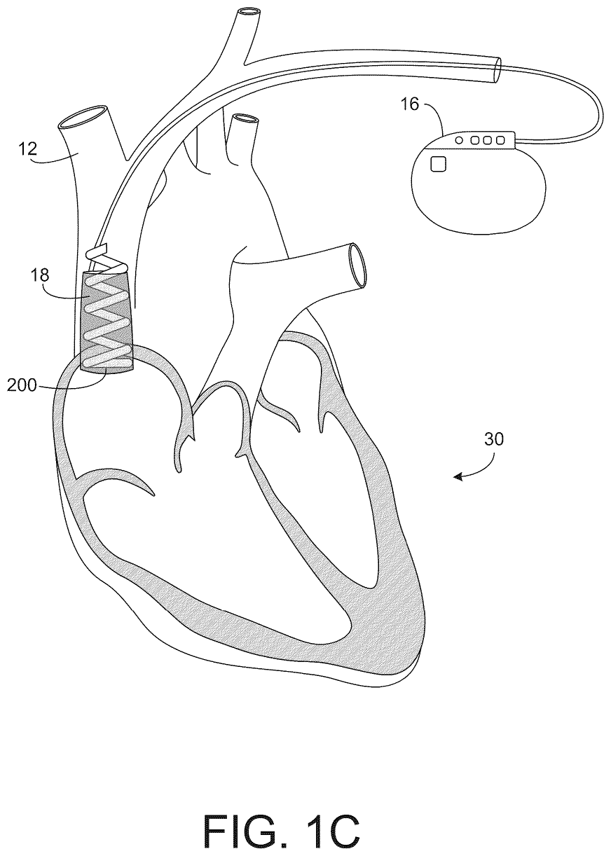

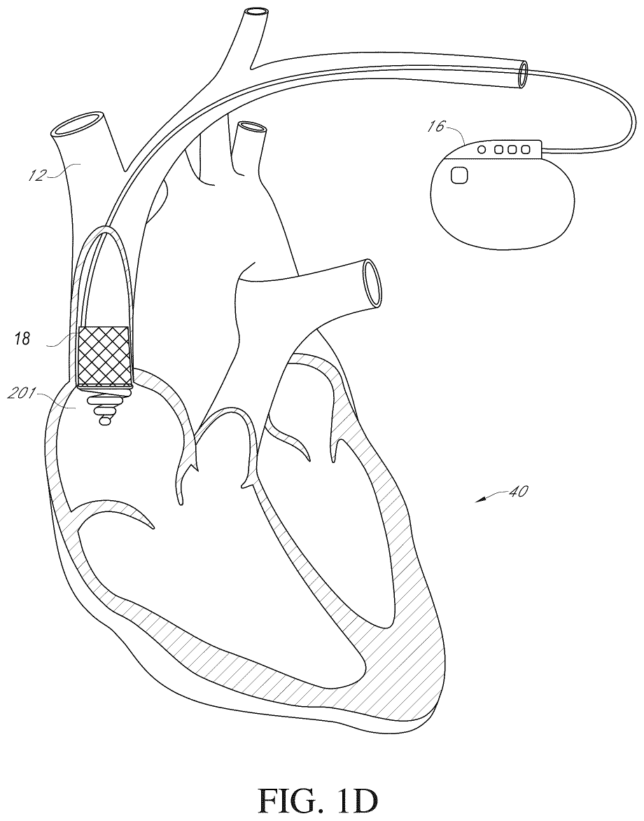

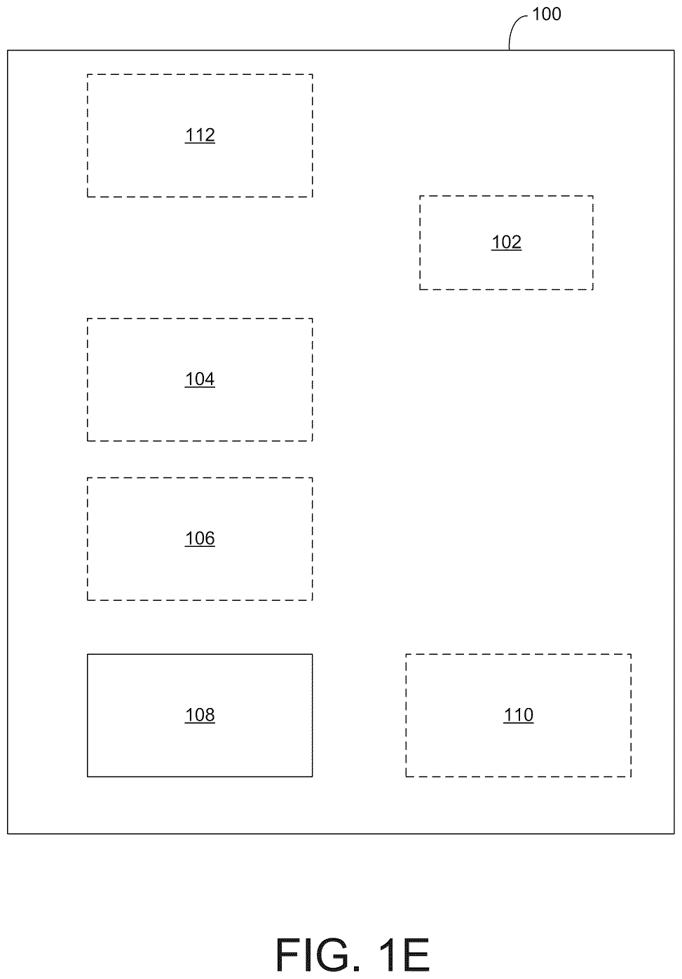

The flow modulating devices described herein may be part of (or installed within) a stent, for example as shown in FIGS. 1B-1C and described in detail below. The stent may represent a frame or outer frame that provides a support structure for the flow regulating devices when the stent is implanted into a blood vessel. The frame/outer frame may be a self-expanding frame or a balloon-expandable frame. In general, any type of stent may be used with the flow regulating devices. Example stents may include, but are not limited to, bare metal stents, coated stents, drug-eluting stents, biodegradable stents, balloon expandable stents, and self-expandable stents.

The stents described herein may be configured to house all or a portion of the flow regulating devices described herein. Such stents may include an assembly with strut members interconnected by joints that form a series of linked mechanisms that result in a hollow substantially tube-shaped element. The stents may be positioned and/or repositioned within a blood vessel to introduce or remove flow regulating devices or device members including, but not limited to, valving, control elements, balloons, flexible members, rigid members, adjustment mechanisms, sensors, coils, wires, and/or magnets. One or more of such device members may be actuated to modify stent shape (or device member shape) or flow regulating device shape (disposed at least partially in stent) for purposes of modifying a flow of fluid through the vessel associated with the implanted stent. Moreover, the stents described herein may partially or fully surround a flow regulating device. For example, a stent or stent portion may surround a portion of a flow regulating device to ensure the device remains in a specified position in a blood vessel. In some examples, the stent surrounds the flow regulating device entirely. In some examples, the stent surrounds the flow regulating device and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricted portions of the stents described herein may result in an outer frame woven tighter than other portions of the stent that are not constricted. The constriction may push against one or more portions of the flow regulating devices described herein to narrow a pathway through the frame or outer frame and/or to trigger the flow regulating device to begin or end constriction. Similarly, expanding portions of the stents described herein may result in an outer frame woven looser than other portions of the stent that are not expanded. The expansion may release one or more portions of the flow regulating devices described herein to widen a pathway through the frame or outer frame and/or to trigger the flow regulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device may be used for implanting devices subcutaneously, for example control devices for controlling activation of any of the flow regulating devices described herein. As described elsewhere herein, various control systems may include an implanted remote device that is configured to transmit control signals to a flow regulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels (e.g., of the lymph, urinary, bile, etc. systems), blood vessels (e.g., superior vena cava, inferior vena cava, renal artery, renal vein, etc.), and/or organ volumes (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example, delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

### Systems and Devices

Disclosed herein are systems and methods for modulating blood flow through a blood vessel. In some examples, the implantable flow modulating devices described herein may be used in blood flow occlusion therapy. For example, the devices described herein may relate to venous occlusion therapy using implantable and/or electronically controlled flow restricting devices for the treatment of acute heart failure. Some devices may be non-implantable or partially implantable. In some examples, the devices described herein generally function to occlude or partially occlude a blood vessel, such as the SVC, the IVC, or related vessels. In some examples, the devices described herein have been contemplated for use in a patient/user having chronic heart failure and/or chronic kidney disease but may be used in any vessel in which flow regulation may be performed.

FIG. 1A illustrates an embodiment of a system 10 for modulating blood flow through a blood vessel. A flow modulating device 200, such as any of the flow modulating devices shown and/or described in FIGS. 1E-8B, is disposed within a blood vessel 12. The flow modulating device 200 includes a valve which is used to restrict blood flow when pressure changes passively cause device actuation (extension, collapse, or intermediate configuration) or certain characteristics (e.g., pressure) are sensed downstream of the device 200. For example, a sensed characteristic may be a right atrium pressure (or other pressure in another vessel), such that increased right atrium pressure (or other pressure in another vessel) may result in flow restriction (i.e., decreasing blood flow) in the blood vessel (e.g., SVC, IVC, etc.), while decreased right atrium pressure (or other pressure in another vessel) may result in flow restriction being alleviated (i.e., returning blood flow to an approximately baseline or original blood flow rate) in the blood vessel. Although a superior vena cava 12 is shown, it is contemplated herein that the systems and devices can be used in any vessel to regulate flow through the vessel.

FIG. 1B illustrates an embodiment of a system 20 for modulating blood flow through a blood vessel. A frame 18, such as a stent or similar device, is disposed within a blood vessel 12. The frame 18 at least partially houses a device 200 (e.g., valve, strip, membrane, etc.) which is used to restrict blood flow when pressure changes passively cause device actuation (extension, collapse, or intermediate configuration) or certain characteristics (e.g., pressure) are sensed downstream of the device 200. For example, a sensed characteristic may be a right atrium pressure (or other pressure in another vessel), such that increased right atrium pressure (or other pressure in another vessel) may result in flow restriction (i.e., decreasing blood flow) in the blood vessel (e.g., SVC, IVC, etc.), while decreased right atrium pressure (or other pressure in another vessel) may result in flow restriction being alleviated (i.e., returning blood flow to an approximately baseline or original blood flow rate) in the blood vessel. A controller 406 may be responsible for causing activation of an actuation device based on blood flow characteristics sensed by one or more sensors positioned to measure the blood flow characteristics, thus actuating device 200. Although a superior vena cava 12 is shown, it is contemplated herein that the systems and devices can be used in any vessel to regulate flow through the vessel.

FIG. 1C illustrates an embodiment of a system 30 for modulating blood flow through a blood vessel. A frame 18, such as a stent or similar device, is disposed within a blood vessel 12. The frame 18 at least partially houses a device 200 (e.g., valve, strip, membrane, etc.) which is used to restrict blood flow when pressure changes passively cause device actuation (extension, collapse, or intermediate configuration) or certain characteristics (e.g., pressure) are sensed downstream of the device 200. For example, a sensed characteristic may be a right atrium pressure (or other pressure in another vessel), such that increased right atrium pressure (or other pressure in another vessel) may result in flow restriction (i.e., decreasing blood flow) in the blood vessel (e.g., SVC, IVC, etc.), while decreased right atrium pressure (or other pressure in another vessel) may result in flow restriction being alleviated (i.e., returning blood flow to an approximately baseline or original blood flow rate) in the blood vessel. A controller 16 may be responsible for causing activation of an actuation device based on blood flow characteristics sensed by one or more sensors positioned to measure the blood flow characteristics, thus actuating of the device 200. Although a superior vena cava 12 is shown, it is contemplated herein that the systems and devices can be used in any vessel to regulate flow through the vessel.

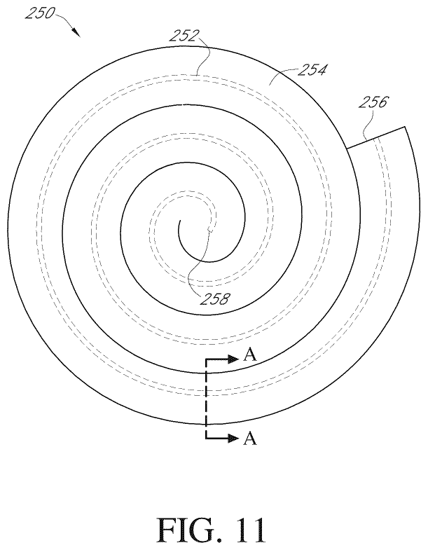

FIG. 1D illustrates an embodiment of a system 40 for modulating blood flow through a blood vessel. A frame 18, such as a stent or similar device, is disposed within a blood vessel 12. The frame 18 at least partially houses a device 201 (e.g., the device 250 of FIGS. 11, 13, and 14) which is used to restrict blood flow when pressure changes passively cause device actuation (extension, collapse, or intermediate configuration) or certain characteristics (e.g., pressure) are sensed downstream of the device 201. For example, a sensed characteristic may be a right atrium pressure (or other pressure in another vessel), such that increased right atrium pressure (or other pressure in another vessel) may result in flow restriction (i.e., decreasing blood flow) in the blood vessel (e.g., SVC, IVC, etc.), while decreased right atrium pressure (or other pressure in another vessel) may result in flow restriction being alleviated (i.e., returning blood flow to an approximately baseline or original blood flow rate) in the blood vessel. A controller 16 may be responsible for causing activation of an actuation device based on blood flow characteristics sensed by one or more sensors positioned to measure the blood flow characteristics, thus actuating of the device 201. Although a superior vena cava 12 is shown, it is contemplated herein that the systems and devices can be used in any vessel to regulate flow through the vessel.

FIG. 1E illustrates a block diagram of an exemplary system 100 for modulating blood flow through a blood vessel. As shown, the system includes an implantable device 108 (e.g., at least a flow occlusion strip, elongate membrane, coilable strip, paddle, conical coiled wire, or expandable cage) and may further optionally include: an optional controller 102, an optional actuation device 104, an optional control element 106, one or more optional sensors 110, and an optional computing device 112. The implantable device 108 may function without actuation devices, sensors, or controllers and may passively actuate (e.g., based on changes in blood flow pressure) between an extended or expanded configuration and a collapsed configuration, or between any intermediate configurations. Alternatively, and optionally (shown by dashed lines), optional actuation device 104 can be coupled to optional control element 106, which may manipulate the implantable device 108 between extended/expanded, collapsed, and/or intermediate configurations. Because the flow occlusion strip in its various configurations described herein functions like a valve, the terms “flow occlusion strip,” “valve,” “spiral valve,” and “elongate membrane,” “coilable strip” may be used interchangeably herein. Optionally (shown by dashed lines), optional controller 102 may be used to control the optional actuation device 104 to actuate the optional control element 106 to manipulate the implantable device 108 between a collapsed configuration (i.e., restricted blood flow position), an extended or expanded configuration (i.e., unrestricted blood flow position), or one or more intermediate configurations therebetween. In passive control embodiments, controller 102, actuation device 104, control element 106, sensor(s) 110, or computing device 112 may not be present. Optional controller 102 may include a microprocessor and any other control devices (e.g., operated switches, motor controllers, antennas, or any other common control devices). The optional controller 102 may optionally receive measurements from one or more optional sensors 110. The one or more optional sensors 110 may measure properties (e.g., pressure) of the blood flowing through a vessel in which the flow modulating device is implanted, or any other physiological or anatomical parameters or properties. Alternatively, or additionally, the optional controller 102 may be preprogrammed with a rule set or predefined parameters that cause the optional controller 102 to activate the optional actuation device based on the rule set and/or predefined parameters.

In operation, the optional controller 102 may cause the optional actuation device 104 to actuate the implantable device 108 (e.g., flex the flow occlusion strip or expand/contract the expandable device) to thereby regulate blood flow through a blood vessel. For example, one or more optional sensors 110 may sense when a pressure exceeds a predefined pressure threshold (e.g., a first pressure state). Based on the detected pressure state, the optional controller 102 may cause the optional actuation device 104 and/or optional control element to manipulate the implantable device 108 (e.g., flex the flow occlusion strip to the collapsed configuration or collapse or compress the expandable device).

For example, the optional actuation device 104 and/or optional control element or passive blood flow (e.g., in the vessel that the device 108 is disposed or in a fluidly connected second vessel) can move the flow occlusion strip such that its surface area is substantially perpendicular to the flow of blood. In such a configuration, the flow occlusion strip may be substantially planar. Further for example, the optional actuation device 104 and/or optional control element or passive blood flow (e.g., in the vessel that the device 108 is disposed or in a fluidly connected second vessel) can move the flow occlusion strip between a substantially flat, coiled shape to an extended spiral shape and back to the substantially, coiled shape (and any intermediate configurations).

For example, the optional actuation device 104 and/or optional control element or passive blood flow (e.g., in the vessel that the device 108 is disposed or in a fluidly connected second vessel) can adjust the expandable cage such that its surface area is substantially perpendicular to the flow of blood. In such a configuration, the expandable cage may be substantially planar. Further for example, the optional actuation device 104 and/or optional control element or passive blood flow (e.g., in the vessel that the device 108 is disposed or in a fluidly connected second vessel) can move the expandable cage between a substantially flat or planar shape to an expanded spherical or ellipsoid shape and any intermediate configurations therebetween.

Similarly, the one or more optional sensors 110 may sense when a pressure drops below a predefined pressure threshold (e.g., a second pressure state). Based on the detected pressure state, the optional controller 102 may cause the optional actuation device 104 to flex the flow occlusion strip to an extended configuration or expand the cage to an expanded configuration.

For example, the actuation device 104 can stretch the flow occlusion strip such that the spirals of its coils are substantially distant from each other, allowing greater blood flow than when in the planar, collapsed configuration. Alternatively, passive pressure changes can cause the flow occlusion strip to be stretched such that the spirals of its coils are substantially distant from each other, allowing greater blood flow than when in the planar, collapsed configuration.

For example, the actuation device 104 can expand the expandable cage such that the leaflets are substantially distant from each other, allowing greater blood flow than when in the planar, collapsed configuration. Alternatively, passive pressure changes can cause the expandable cage to be expanded such that the leaflets are substantially distant from each other, allowing greater blood flow than when in the planar, collapsed configuration.

When a pressure between the first and second pressure states (e.g., a third pressure state) is sensed by the optional one or more sensors 110, the optional controller 102 may cause the actuation device 104 and/or control element to flex the flow occlusion strip or expand the cage (device 108) to one or more intermediate configurations. Alternatively, passive pressure changes can cause the flow occlusion strip or cage (device 108) to be stretched to one or more intermediate configurations. An intermediate configuration can be any configuration in which the flow occlusion strip or cage (device 108) is stretched beyond the substantially planar shape of the collapsed configuration but less so than the maximum extension of the extended or expanded configuration. Optionally, an optional antenna (not shown) disposed in, or within proximity of, the optional controller 102 may be used to transmit data to an optional remote computing device 112, for example a server, workstation, physician computing device, or mobile computing device. The transmitted data may include sensor 110 readings, flow occlusion strip position, actuation events, or any other data from the system. Data processing may occur locally on the optional controller 102 and/or remotely on the optional remote computing device 112. Additionally, the optional one or more sensors 110 need not be adjacent or physically coupled to the implantable device 108, optional controller 102, or optional actuation device 104. In some embodiments, the one or more optional sensors 110 can be located in a different body portion (e.g., different blood vessel), than that of the flow occlusion strip or cage (device 108) when implanted in a body.

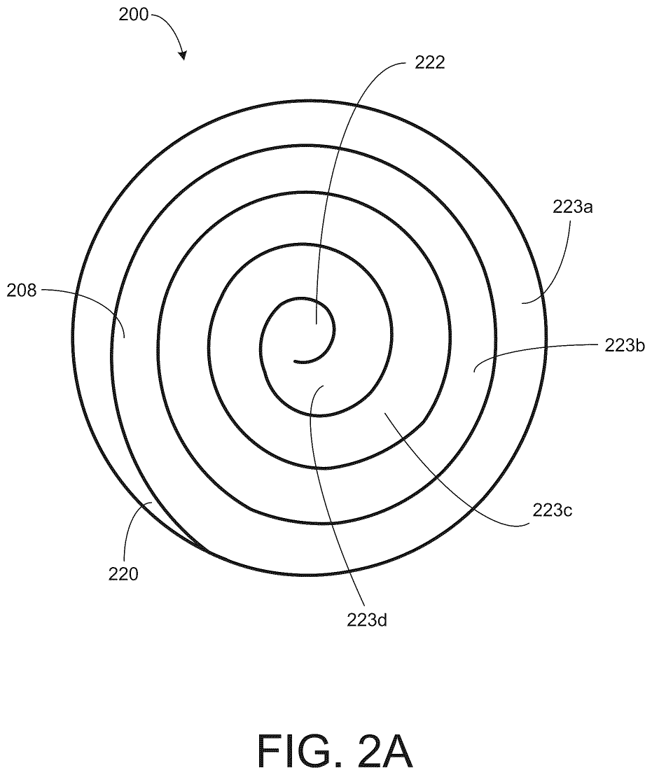

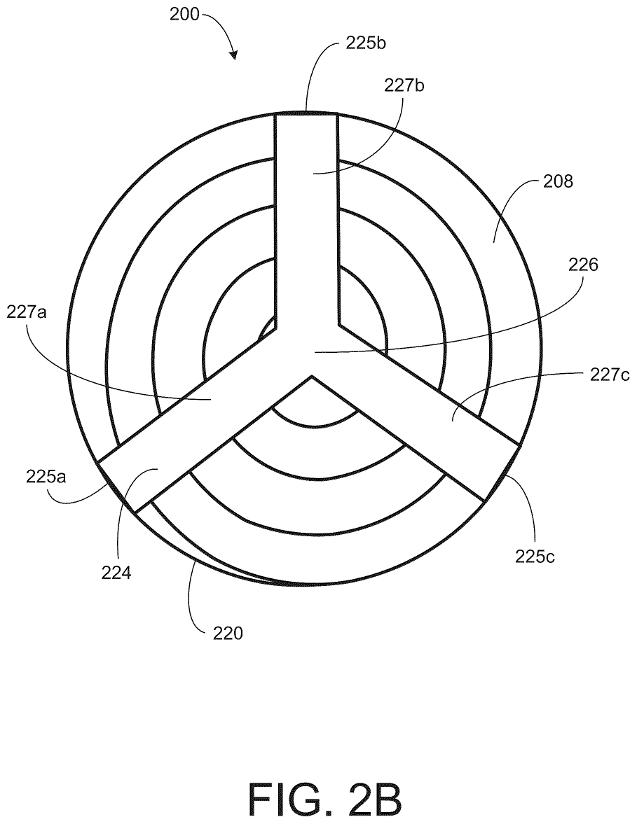

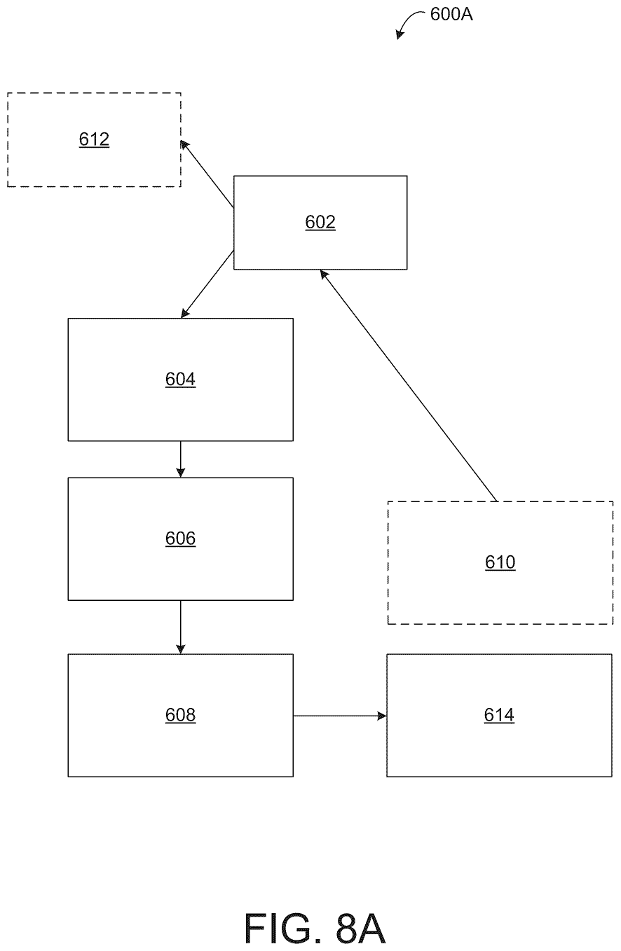

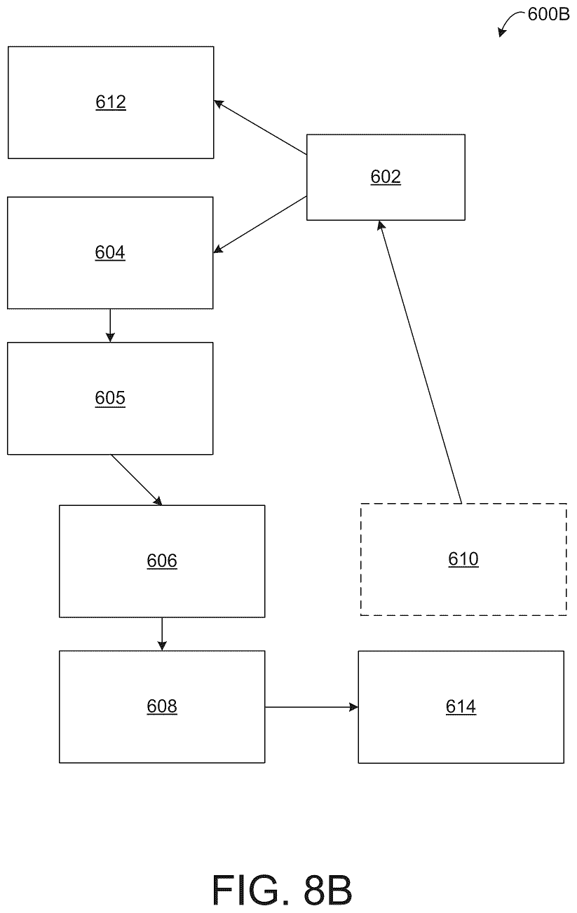

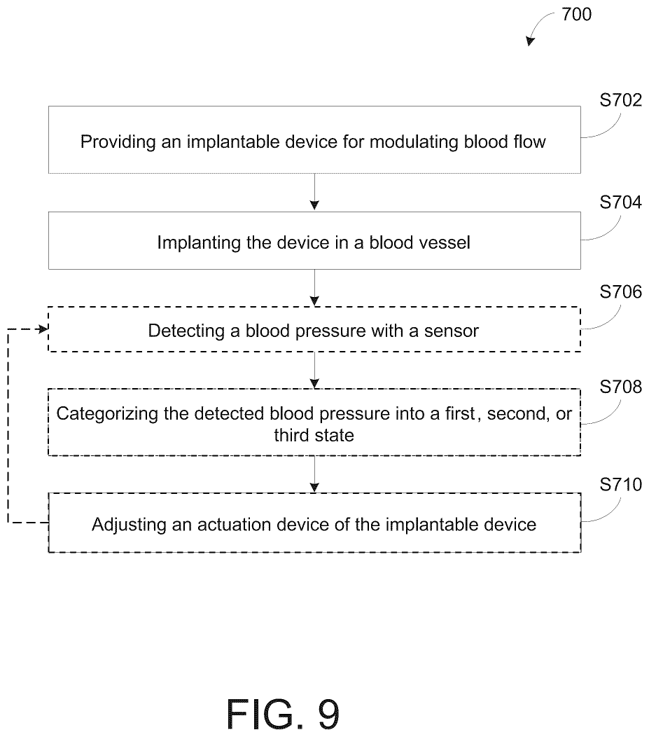

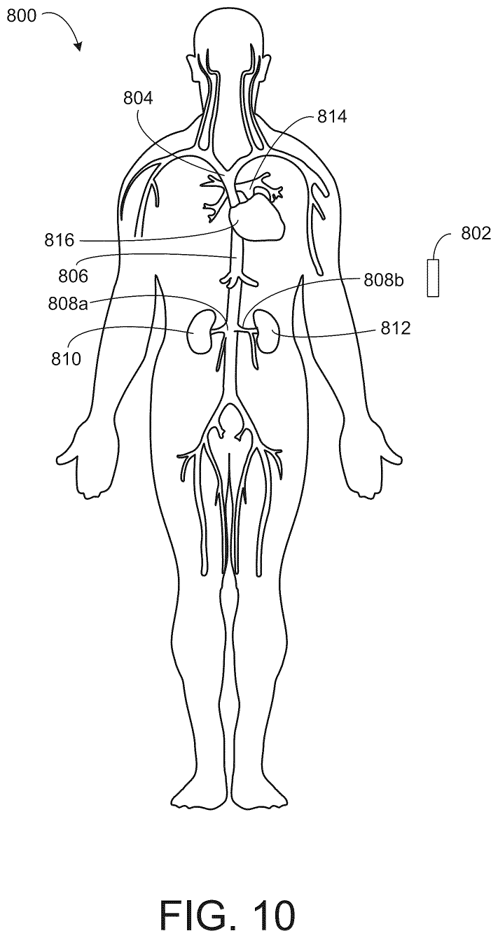

FIGS. 2A-5 illustrate various embodiments of implantable devices for modulating flow through a vessel. For example, the vessel may be a blood vessel, although urinary, bile, lymphatic, etc. vessels are also contemplated herein. The fluid may be blood, but may also be bile, urine, lymph, etc. without departing from the scope of the present disclosure. The devices shown and described in FIGS. 2A-5, 6A-6B, and 7A-7C may be used in the systems of FIGS. 1E and 8A-8B and the method of FIGS. 9-10, as described elsewhere herein.

FIG. 2A illustrates a top view of an example embodiment of a device 200 for modulating blood flow in a blood vessel. The device 200 includes an elongate membrane 208 shaped in a spiral (e.g., coiled) configuration or shape having a first end 220 and a second end 222. The elongate membrane 208 may have a substantially ribbon-like cross-section, a substantially elliptical or oval cross-section, or a substantially circular cross-section. The elongate membrane 208 is adapted to flex between a collapsed configuration (i.e., a restricted blood flow position) and an extended configuration (i.e., an unrestricted blood flow position), transitioning between any one or more intermediate configurations. In FIG. 2A, the elongate membrane 208 is depicted in the collapsed configuration. In some embodiments, the elongate membrane 208 is further adapted to flex to or one or more intermediate positions between the collapsed configuration and the extended configuration.

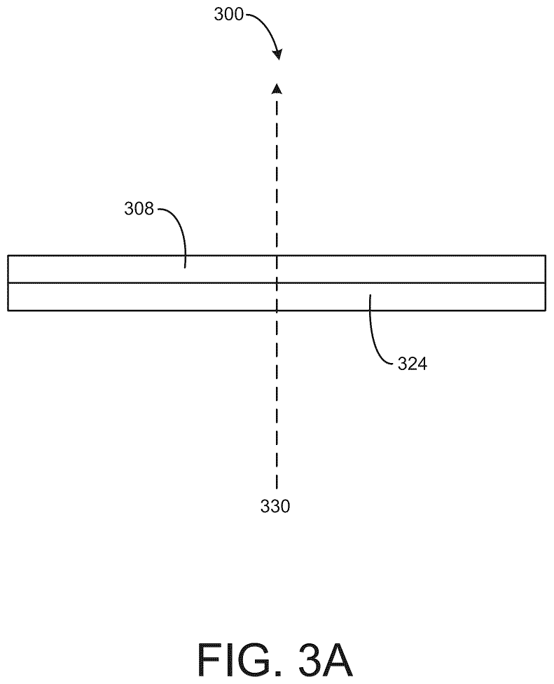

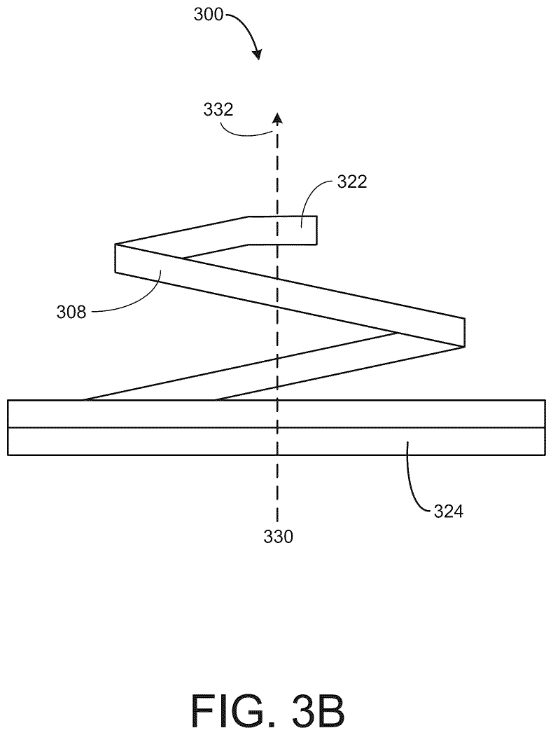

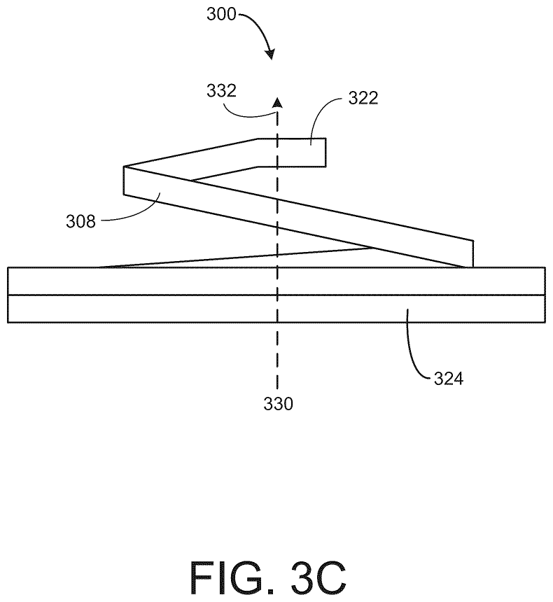

In the collapsed configuration, as shown in FIG. 2A, the coils 223a, 223b, 223c, 223d . . . 223n of the elongate membrane 208 are sufficiently close together as to be substantially planar (see, e.g., FIG. 3A for a side view of the collapsed configuration). For example, coil 223d may be nested in coil 223c, which may be nested in coil 223b, which may be nested in coil 223a. Said another way, coil 223d may rest in an inner perimeter of coil 223c, which may rest in an inner perimeter of coil 223b, which may rest in an inner perimeter of coil 223a. As shown in FIG. 2A, the coils 223a, 223b, 223c, 223d are continuous with one another. Alternatively, each coil may form a separate ring, with the rings being coupled to one another by a coupling element, such as a scalloped line, suture, leaflet, or the like. When implanted in a blood vessel, the collapsed configuration leaves little to no space between its coils, therefore occluding the majority of the blood vessel and allowing very little to no blood to flow therethrough. In some embodiments, the collapsed configuration can occlude about 80% or more of the surface area of a cross-section of the blood vessel in which device or membrane 208 is implanted. In some embodiments, the collapsed configuration can occlude about 90% or more of the surface area of a cross-section of the blood vessel in which it's implanted. In some embodiments, the collapsed configuration can occlude substantially 100% of the surface area of a cross-section of the blood vessel in which it's implanted. In some embodiments, the collapsed configuration can reduce a blood flow rate through the elongate membrane 208 by about 80% or more when compared to a blood flow rate through an analogous section of the relevant blood vessel lacking the implanted device 200. In some embodiments, the collapsed configuration can reduce a blood flow rate through the elongate membrane 208 by about 90% or more. In some embodiments, the collapsed configuration can reduce a blood flow rate through the elongate membrane 208 by substantially 100%.

FIG. 2B illustrates a bottom view of an example embodiment of device 200. The device 200 includes a support frame 224 coupled to the elongate membrane 208. The support frame 224 includes a hub 226 (which may be centered on elongate membrane 208 or offset) having one or more arms 227 extending therefrom. Although three arms 227 are shown, one of skill in the art will appreciate that one arm, one or more arms, two or more arms or a plurality of arms may be present. A first arm 227a may terminate at a first arm end 225a, an optional second arm 227b may terminate at a second arm end 225b, and an optional third arm 227c may terminate at a third arm end 225c. The support frame 224 may have at least one arm end 225a, 225b, and/or 225c near (e.g., substantially adjacent to or proximal to) the second end 222. The term “near” in this context should be understood to mean closer to the first end 220 than to the second end 222 along a linear length of the spiral shape of the elongate membrane 208. In the embodiment of FIG. 2B, the arm end 225a is near to the first end 220. In addition, the support frame 224 is coupled to the elongate membrane 208 at each of the three arm ends 225a, 225b, and 225c. Alternatively, as described above, the support frame 224 can be coupled to the elongate membrane 208 at one arm end, one or more arm ends, two or more arm ends, or a plurality of arm ends. In some examples, second end 222 is extendable and collapsible relative to the support frame 224 and along a longitudinal access (see, e.g., FIG. 3A below).

FIG. 3A illustrates a side view of an embodiment of a device 300 for modulating blood flow through a blood vessel. The device 300 is shown in a collapsed configuration with coilable strip 308 at least approximately flush with support frame 324 and/or a majority or two or more of the coils (shown in FIG. 2A) may be at least partially in contact with support frame 324. In some embodiments, the second end 222 (see, e.g., FIG. 2A) is extendable and collapsible relative to the support frame 324 and along longitudinal axis 330. When the device 300 is implanted in a blood vessel, longitudinal axis 330 is parallel to the direction of blood flow in the blood vessel, in some embodiments. Put another way, when the device 300 is implanted in a blood vessel, longitudinal axis 330 is perpendicular to a cross-sectional area of the blood vessel and the device 300. This can allow for the second end 222 (see, e.g., FIG. 2B) to extend in the same direction as the blood flow direction in the blood vessel in some embodiments. In some embodiments, this can allow for the second end 222 (scc, e.g., FIG. 2B) to extend in the opposite direction as the blood flow direction in the blood vessel.

FIG. 3B illustrates a side view of an embodiment of the device 300 in an extended configuration. The second end 322 of the coilable strip 308 is extended away from the support frame 304 in a stretched (e.g., elongated) spiral shape around the longitudinal axis 330 and moving along the longitudinal axis 330 in the direction of arrow 332. As the coilable strip 308 is flexed (e.g., stretched, unwound, uncoiled, etc.) from the collapsed configuration into the extended configuration, the spiral shape of coilable strip 308 is revealed and allows a higher rate of blood flow through the device 300 in comparison to the collapsed configuration but less than or equal to a blood flow rate through an analogous blood vessel lacking the device 300. In some embodiments, the extended configuration can reduce a blood flow rate through the coilable strip 308 by about 0% to about 30% when compared to a blood flow rate through an analogous section of the relevant blood vessel lacking the implanted device 300. In some embodiments, the extended configuration can reduce a blood flow rate through the coilable strip 308 by about 0% to about 20% when compared to a blood flow rate through an analogous section of the relevant blood vessel lacking the implanted device 300. In some embodiments, the extended configuration can reduce a blood flow rate through the coilable strip 308 by about 0% to about 10% when compared to a blood flow rate through an analogous section of the relevant blood vessel lacking the implanted device 300.

The device 300 can also flex to one or more intermediate positions, as shown in FIG. 3C, between a collapsed configuration (e.g., FIG. 3A) and an extended (e.g., FIG. 3B) configuration. Intermediate positions (see, e.g., FIG. 3C) can be measured by a distance of the second end 322 from the support frame 304 along the longitudinal axis 330. Intermediate positions bring the coils of the coilable strip 308 closer together in comparison to the extended configuration but not to the degree of the substantially planar arrangement of the collapsed configuration. Therefore, intermediate positions can reduce a blood flow rate through the coilable strip 308 when compared to a blood flow rate through an analogous section of the relevant blood vessel lacking the implanted device 300 to an extent greater than the extended configuration but less than that of the collapsed configuration. In some embodiments, the intermediate positions can reduce a blood flow rate through the coilable strip 308 by about 5% to about 95% when compared to a blood flow rate through an analogous section of the relevant blood vessel lacking the implanted device 300. In some embodiments, the intermediate positions can reduce a blood flow rate through the coilable strip 308 by about 10% to about 90% when compared to a blood flow rate through an analogous section of the relevant blood vessel lacking the implanted device 300. In some embodiments, the intermediate positions can reduce a blood flow rate through the coilable strip 308 by about 20% to about 80% when compared to a blood flow rate through an analogous section of the relevant blood vessel lacking the implanted device 300.

In some embodiments, the device 300 is adapted to flex between a collapsed configuration, an extended configuration, and/or one or more intermediate positions between the collapsed configuration and the extended configuration. The device 300 is adapted to flex between these configurations by incorporating appropriate materials (e.g., tissue, sutures, fabric, elastics, etc.), appreciated by those of skill in the art, that can stretch or bend without fracturing or torquing either passively according to a blood pressure or other physiological state acting on the device or by active mechanisms, as described herein. The flex may be performed passively or automatically in response to a local blood pressure (blood pressure in the same vessel in which the device is implanted) in the blood vessel acting on the device 300 when implanted. For example, in flexing (e.g., in response to passive blood pressure or flow changes or an active actuation mechanism), second end 322 of the device 300 may be offset or moved away, shown by arrow 332, from frame 324 and/or first end 220 (shown in FIG. 2B), along longitudinal axis 330.

In some variations, the device 300 comprises Nitinol®, such that the Nitinol® is shape set (i.e., pre-trained shape memory) to the substantially collapsed or planar configuration, such that, in the absence of a passive pressure change or an active actuation mechanism, the device 300 may remain in the substantially collapsed or planar configuration. Alternatively, device 300 may be shape set (i.e., pre-trained shape memory) to the substantially extended or spiral configuration, such that, in the absence of a passive pressure change or an active actuation mechanism to move the device to the collapsed configuration or one or more intermediate positions, the device 300 may remain in the substantially extended configuration.

As described above, the support frame 324 and the coilable strip 308 can be adapted such that under various ranges of blood pressures experienced by the device 300 when implanted, the coilable strip extends or collapses accordingly. In some embodiments, this passive response is achieved by constructing the coilable strip 308 and support frame 324 of appropriate materials, for example Nitinol®, Stainless Steel, Cobalt Chromium, and the like. In some embodiments, the coilable strip 308 may be implanted in a subject and can be adapted to collapse or be compressed when a blood pressure of the subject (e.g., right atrial pressure) is determined or detected to be, for example, about 30 mmHg or less, about 25 mmHg or less, or about 20 mmHg or less (i.e., within a first range of blood pressures). The implanted coilable strip 308 may be adapted to extend when a blood pressure of the subject is determined to be, for example, about 25 mmHg or higher or about 30 mmHg or higher (i.e., within a second range of blood pressures). In this embodiment, the implanted coilable strip 308 can flex to one or more intermediate positions when the blood pressure of the subject is determined to be between about 10 mmHg and about 30 mmHg (i.e., within a third range of blood pressures). In some variations, a flow modulating device (e.g., any of devices of FIGS. 1A-8B) may respond to pressure indirectly. For example, the flow modulating device (e.g., any of devices of FIGS. 1A-8B) may modulate flow through a vessel in response to a change in diameter of the vessel, which may correlate to a predefined pressure range.

In some variations, an implanted flow modulating device (e.g., any of devices of FIGS. 1A-8B) may create a pressure gradient across the device of about 5 mmHg, greater than about 5 mmHg, or between about 3 mmHg and about 10 mmHg. In one non-limiting example, when attempting to maintain right atrial pressure below about 10 mmHg, and right atrial pressure rises to about 12 mmHg or 13 mmHg, implanting a flow modulating device (e.g., any of devices of FIGS. 1A-8B) with a gradient of about 5 mmHg may result in an upstream right atrial pressure of about 15 mmHg and downstream right atrial pressure may be maintained at about 10 mmHg.

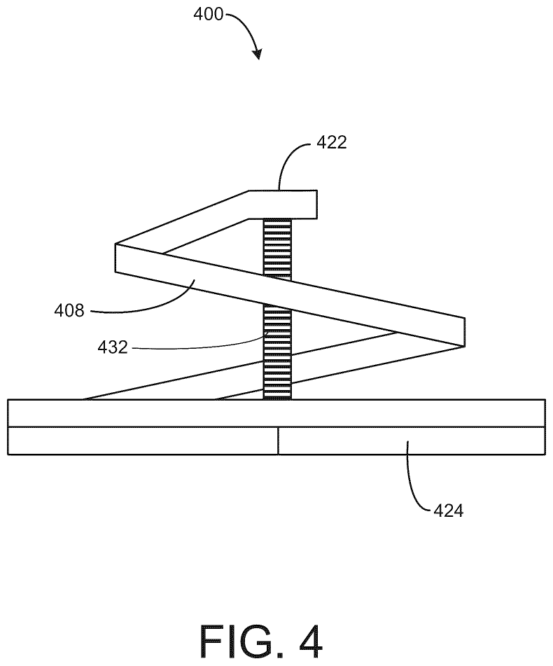

FIG. 4 illustrates a side view of an embodiment of a device 400 for modulating blood flow in a blood vessel. As shown in FIG. 4, the device 400 further includes a tensioner 432 coupled to the support frame 424 and the second end 422. In this embodiment, the tensioner 432 is adapted to maintain the flow occlusion strip 408 in the collapsed configuration under a first range of blood pressures, maintain the flow occlusion strip in the extended configuration under a second range of blood pressures, and maintain the flow occlusion strip in one or more intermediate positions under a third range of blood pressures between the first and second ranges. In some embodiments, the tensioner 432 can include a linear actuator, for example, a spring or piston, as described herein. For example, the linear actuator, acting as the tensioner 432, can apply enough resistance to the flow occlusion strip 408 to maintain the flow occlusion strip in the collapsed position at a blood pressure of, for example, about 30 mmHg or less, about 25 mmHg or less, or about 20 mmHg or less (i.e., within a first range of blood pressures). The linear actuator, acting as the tensioner 432, can apply enough resistance to maintain the flow occlusion strip 408 in the extended configuration at a blood pressure of, for example, about 25 mmHg or higher or about 30 mmHg or higher (i.e., within a second range of blood pressures). The spring or piston, acting as the tensioner 432, can apply enough resistance to maintain the flow occlusion strip 408 in one or more intermediate positions at blood pressures between about 10 mmHg and about 30 mmHg (i.e., within a third range of blood pressures).

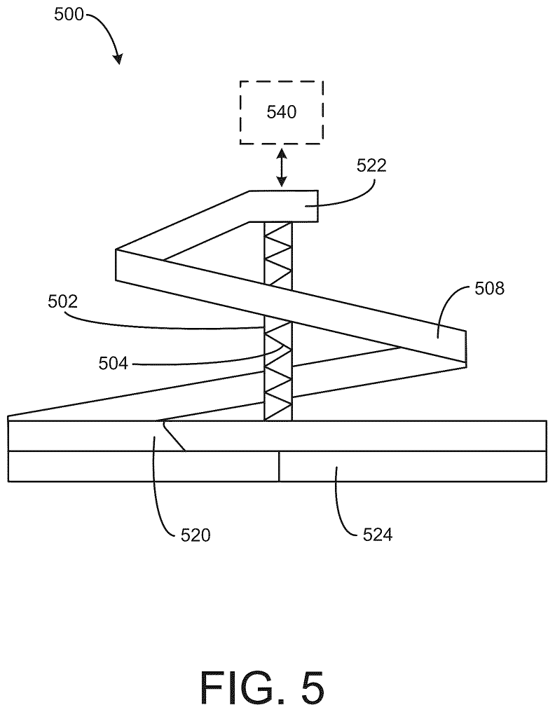

FIG. 5 illustrates a side view of an embodiment of a device 500 for modulating blood flow in a blood vessel. As shown in FIG. 5, the device 500 includes a control element 502 coupled to the support frame 524 at the first strip end 520 of the flow occlusion strip 508. The control element 502 is adapted to maintain the flow occlusion strip in one or more of: the collapsed configuration, the extended configuration, or the one or more intermediate positions. In some embodiments, the control element 502 adapted to reversibly maintain the flow occlusion strip in one or more of: the collapsed configuration, the extended configuration, or the one or more intermediate positions. In some embodiments, the control element 502 further includes a linear actuator 504 configured to tension the control element to maintain the flow occlusion strip in one or more of: the collapsed configuration, the extended configuration, or the one or more intermediate positions.

In some embodiments, the linear actuator 504 includes a material having shape memory or pseudo elastic properties. As appreciated by those of skill in the art, shape memory is the property of a material to exhibit a first shape under a first temperature but adopt a second, predetermined shape under a second temperature. As appreciated by those of skill in the art, pseudo elasticity is a property in which a material can reversibly transform between two predetermined shapes in response to the presence or absence of a known quantity of physical stress. Many shape memory alloys have pseudo clastic properties. Examples of shape memory alloys include Nitinol®. In some embodiments, the linear actuator 504 includes Nitinol®. In some embodiments, the control element 502 with or without the linear actuator 504 allows for the active management of blood flow modulation, as described herein. In some examples, the active management can be achieved by applying a temperature or electric current to a linear actuator 504 comprising Nitinol® or other shape memory alloy to cause deformation of the Nitinol® to transition between an extended (i.e., elongate) and retracted (i.e., shorten) state.

Alternatively, or additionally, an optional actuation device 540 may be used to tension or relax control element 502 to extend or collapse, respectively, device 500 or position device 500 at an intermediate configuration,

In some embodiments, the device 500 can include an optional sensor 110 (see, e.g., FIG. 1E), a power source (not shown), and a microcontroller 102 (see, e.g., FIG. 1E) electrically coupled to the linear actuator 504 and the optional sensor 110. In the depicted embodiment of FIG. 5, the linear actuator 504 is configured to tension the control element 502 to maintain the flow occlusion strip 508 in an extended configuration in response to a first pressure state sensed by the sensor; to release tension in the control element 502 to maintain the flow occlusion strip 508 in a collapsed configuration in response to a second pressure state sensed by the senor; and to tension the control element 502 to maintain the flow occlusion strip 508 in one or more intermediate positions in response to a third range of pressure states sensed by the sensor that is between the first and second pressure state. In some embodiments, the first pressure state can be a pressure range of a blood pressure of, for example, about 30 mmHg or less, about 25 mmHg or less, or about 20 mmHg or less. In some embodiments, the second pressure state can be a pressure range of a blood pressure of, for example, about 25 mmHg or higher or about 30 mmHg or higher. In further embodiments, the third pressure state can be a pressure range of a blood pressure between about 10 mmHg and about 30 mmHg.

In some embodiments, the controller 102 and remote computing device 112 of FIG. 1E can assist in the determination of first, second, and third pressure states as sensed by the sensor. For example, the sensor 110 of FIG. 1E can detect a blood pressure and transmits the sensed blood pressure to controller 102. The controller 102 may process the blood pressure locally or may optionally transmit the detected blood pressure to a remote computing device 112. The remote computing device then determines whether the detected pressure value falls into the first, second, or third pressure state and transmits its determination back to the controller 102. The controller 102 then activates the actuation device 104 to flex the flow occlusion strip or manipulate the expandable cage to the corresponding configuration or intermediate position.

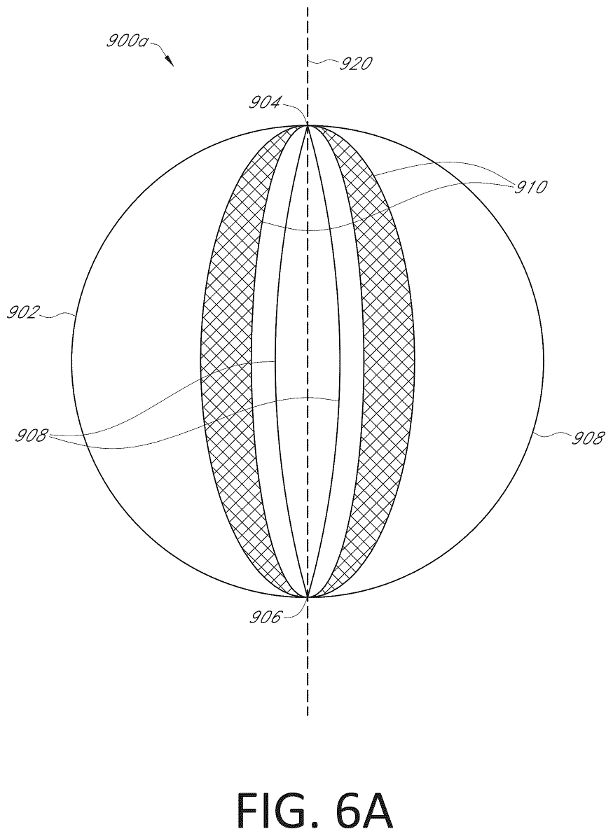

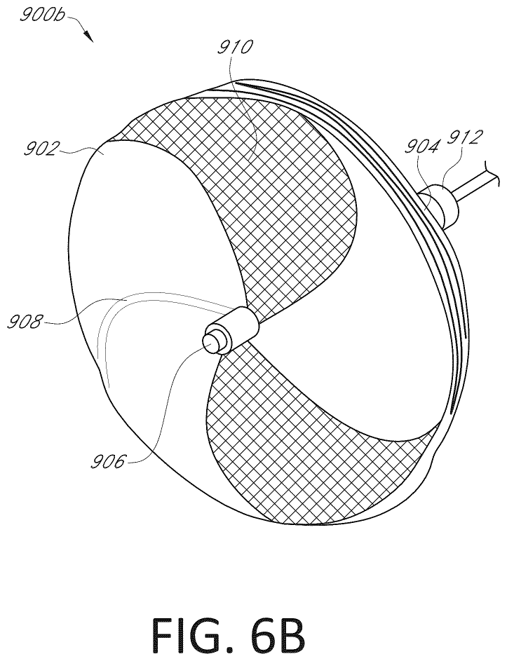

FIGS. 6A-6B illustrate another embodiment of an implantable device 900a in an expanded configuration and the implantable device 900b in a collapsed configuration. The device 900a, 900b may be used in the systems of FIGS. 1E and 8A-8B and the method of FIGS. 9-10, as described elsewhere herein. The device is configured to be implanted in a vessel, for example a blood vessel, to modulate a flow of fluid (e.g., blood) through the vessel. The vessel may be an inferior vena cava or a superior vena cava, for example. The implantable device may be delivered to the vessel using any of the delivery systems described elsewhere herein. As shown in FIG. 6A, the implantable device 900a includes an expandable cage 902 having a first end 904 opposite a second end 906. The expandable cage 902 may include or be formed of wire 908, for example Nitinol®. The Nitinol® may be shape set (i.e., pre-trained shape memory) into the expanded configuration or the collapsed configuration, such that the Nitinol® is biased toward the expanded configuration or the collapsed configuration, respectively. The expandable cage 902 can include two or more wires 908 connecting the first end 904 to the second end 906. Alternatively, the expandable cage 902 can include a plurality of wires 908 connecting the first end 904 to the second end 906. The cage 902 and/or wires 908 may be coated with a polymer, drug, textile, tissue, and/or the like. The implantable device 900a further includes one or more leaflets 910 extending between the first end 904 and the second end 906. The leaflets 910 may comprise or be composed of a textile, tissue, polymer, etc. as indicated elsewhere herein.

As shown in FIGS. 6A-6B, the expandable cage 902 is movable between a collapsed configuration (device 900b shown in FIG. 6B), in which the one or more leaflets 910 are configured to at least partially prolapse, and an expanded configuration (device 900a shown in FIG. 6A), in which the one or more leaflets 910 are configured to elongate or extend between the first end 904 and second end 906. When the one or more leaflets 910 are in the collapsed or prolapsed configuration, the one or more leaflets 910 at least partially occlude the expandable cage (thereby at least partially occluding the blood vessel in which the implantable device is disposed). Although an expanded configuration (device 900a shown in FIG. 6A) and collapsed configuration (device 900b shown in FIG. 6B) of cage 902 are shown, one or more intermediate configurations between the collapsed configuration and the expanded configuration are also possible.

Further, as shown in FIG. 6A, the wire(s) 908 have an arcuate shape in the expanded configuration (device 900a). In the collapsed configuration (device 900b), shown in FIG. 6B, the wire(s) 908 have a partial spring or spiral shape The device 900a, 900b may further include an optional lock 912 (shown in FIG. 6B) on the first end 904 or the second end 906. The optional lock 912 maintains the implantable device in the expanded configuration (device 900a shown in FIG. 6A). The optional lock 912 may be manipulatable by an actuation device, as described elsewhere herein, such that the actuation device releases the optional lock 912 to collapse or expand the device or engages the optional lock 912 to fix the device in a configuration (e.g., collapsed, expanded, intermediate). Exemplary, non-limiting examples of lock 912 include a linear ratchet and pawl lock, a spring lock, a rotary lead screw with powered motor, and the like. Alternatively, or additionally, a control element, as described elsewhere herein, may be coupled to the cage 902 (e.g., at first end 904 or second end 906) or optional lock 912 to cause the cage 902 to be manipulated between the expanded (device 900a shown in FIG. 6A) and collapsed (device 900b shown in FIG. 6B) configuration. Further still, cage 902 may optionally be disposed in a frame, such as a stent, for implantation in a vessel, as described elsewhere herein. Expandable cage 902 may be implanted in a vessel with a longitudinal axis 920 of the cage 902 perpendicular to or, alternatively, parallel with a longitudinal axis of the vessel in which the device is implanted. Said another way, expandable cage 902 may be implanted in a vessel with a longitudinal axis 920 of the cage 902 perpendicular to or, alternatively, parallel with a flow of fluid in the vessel in which the device is implanted.

As described above, the cage 902 can be adapted such that under various ranges of blood pressures experienced by the device 900a, 900b when implanted, the cage 902 expands or collapses accordingly. In some embodiments, this passive response is achieved by constructing the cage 902 of appropriate materials, for example Nitinol®, Stainless Steel, Cobalt Chromium, and the like. In some embodiments, the cage 902 may be implanted in a subject and can be adapted to collapse or be compressed when a blood pressure of the subject is determined or detected to be, for example, about 30 mmHg or less, about 25 mmHg or less, or about 20 mmHg or less (i.e., within a first range of blood pressures). The implanted cage 902 may be adapted to expand when a blood pressure of the subject is determined to be, for example, about 25 mmHg or higher or about 30 mmHg or higher (i.e., within a second range of blood pressures). In this embodiment, the implanted cage 902 can flex to one or more intermediate positions when the blood pressure of the subject is determined to be between about 10 mmHg and about 30 mmHg (i.e., within a third range of blood pressures).

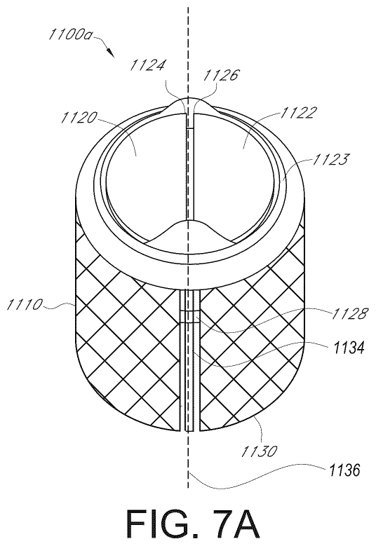

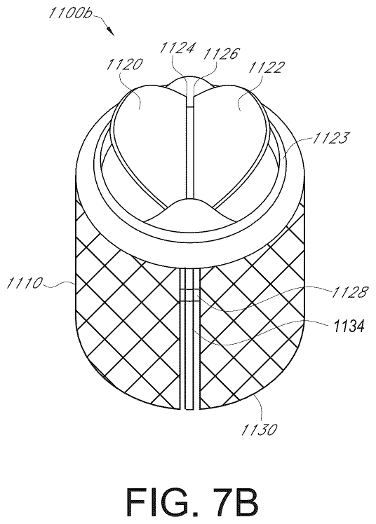

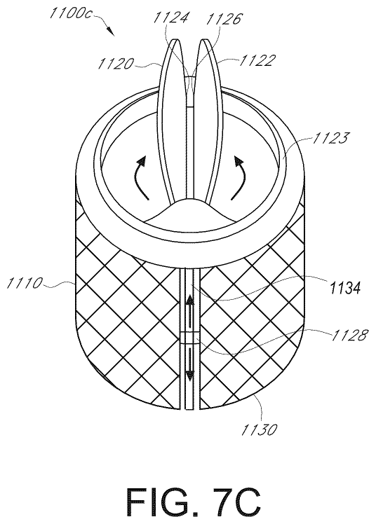

FIGS. 7A-7C show another embodiment of a flow modulating device 1100 for modulating blood flow through a blood vessel. The flow modulating device 1100a is shown in a closed configuration in FIG. 7A, a partially open configuration (device 1100b also described herein as a partially closed configuration) in FIG. 7B, and an open configuration (device 1100c also described herein as a fully open configuration) in FIG. 7C. The flow modulating device 1100a, 1100b, 1100c includes a frame 1110 including one or more paddles 1120, 1122 pivotally coupled to the frame 1110 at one or more pivot points 1124, 1126, respectively. Frame 1110 defines an inflow end 1130 and an outflow end 1123, although the opposite is also conceived. Fluid flowing through the vessel in which the device 1100a, 1100b, 1100c is implanted flows into the frame 1110 through inflow end 1130 and flows out of the device 1100a, 1100b, 1100c through outflow end 1123. The one or more paddles 1120, 1122 reduce flow through the frame 1110, for example in the closed (device 1100a shown in FIG. 7A) or partially open configuration (device 1100b shown in FIG. 7B) and permits substantial flow through the frame 1110 in the open configuration 1110c. The frame 1110 may further include rail or shaft 1134 on which stop 1128 translates to permit or increase a degree of rotation of paddles 1120, 1122 relative to frame 1110 or reduce a degree of rotation of paddles 1120, 1122 relative to frame 1110. For example, stop 1128 may translate on rail or shaft 1134 toward the inflow end 1130 to permit paddles 1120, 1122 to pivot until the one or more paddles 1120, 1122 are substantially parallel to a longitudinal axis 1136 of frame 1110, as shown in FIG. 7A. Alternatively, stop 1128 may translate on rail or shaft 1134 toward the outflow end 1123 to permit paddles 1120, 1122 to pivot until the one or more paddles 1120, 1122 are either angled relative to a longitudinal axis 1136 of the frame 1110 (as shown in FIG. 7B) or substantially perpendicular to a longitudinal axis 1136 of the frame 1110 (as shown in FIG. 7C). Translation of stop 1128 toward the inflow end 1130 or the outflow end 1123 causes rotation of paddles 1120, 1122 and therefore adjusts an amount of blood flow through frame 1110. Alternatively, or additionally, frame 1110 may include a translation mechanism on which stop translates. Stop 1128 may be caused to translate on shaft 1134 via a passive mechanism (e.g., pullwire, cable, etc.) or via an active mechanism (e.g., magnetic actuator, linear actuator, etc.), examples of each of which are described elsewhere herein.

In some embodiments, the one or more paddles 1120, 1122 may be pivoted to a closed configuration (device 1100a shown in FIG. 7A) or a partially open configuration (device 1100b shown in FIG. 7B) when a blood pressure of the subject is determined or detected to be, for example, about 30 mmHg or less, about 25 mmHg or less, or about 20 mmHg or less (i.e., within a first range of blood pressures). The one or more paddles 1120, 1122 may be pivoted to an open configuration (device 1100c shown in FIG. 7C) when a blood pressure of the subject is determined to be, for example, about 25 mmHg or higher or about 30 mmHg or higher (i.e., within a second range of blood pressures). Further, in some variations, the one or more paddles 1120, 1122 can be pivoted to one or more intermediate positions (see, e.g., the partially open configuration shown as device 1100b in FIG. 7B) when the blood pressure of the subject is determined to be between about 10 mmHg and about 30 mmHg (i.e., within a third range of blood pressures).

Said another way, the stop 1128 may be translated toward the outflow end 1123 to cause the paddles 1120, 1122 to move to a closed configuration (device 1100a shown in FIG. 7A) or a partially open configuration (device 1100b shown in FIG. 7B) when a blood pressure of the subject is determined or detected to be, for example, about 30 mmHg or less, about 25 mmHg or less, or about 20 mmHg or less (i.e., within a first range of blood pressures). The stop 1128 may be translated toward the inflow end 1130 to cause the paddles 1120, 1122 to move to an open configuration (device 1100c shown in FIG. 7C) when a blood pressure of the subject is determined to be, for example, about 25 mmHg or higher or about 30 mmHg or higher (i.e., within a second range of blood pressures). Further, in some variations, the stop 1128 may be translated toward the outflow end 1123 to cause the paddles 1120, 1122 to move to one or more intermediate positions (see, e.g., the partially open configuration, shown as device 1100b in FIG. 7B) when the blood pressure of the subject is determined to be between about 10 mmHg and about 30 mmHg (i.e., within a third range of blood pressures).

FIG. 8A depicts another embodiment of a system 600A for modulating blood flow through a blood vessel. The system 600A includes a first magnet 606, an actuation device 608, and an elongate member 614. The first magnet 606 is operatively coupled to the actuation device 608, and the actuation device 608 is operatively coupled (e.g., at the second end 322 of FIG. 3B) to the elongate member 614 to effect movement of the elongate member 614. The system 600A may further include a control device 602 operatively coupled to a second magnet 604. The control device 602 can include a microprocessor, power source (e.g., a battery, a capacitor, wall outlet, or any other suitable power source), antenna, operated switches, and/or any other control devices. The control device 602 and second magnet 604 may be located externally, but proximal to a user. Alternatively, the control device 602 and second magnet 604 may be implanted (e.g., subcutancously, intravascularly, etc.). Optionally, the system 600A may include an optional sensor 610. The optional sensor 610 may sense one or more physiological or anatomical attributes and output a signal to the control device 602, which may output an activation signal to the actuation device 608 to expand or collapse the elongate member or move the elongate member 614 to one or more intermediate configurations.

The actuation device 608 may be a linear actuator coupled to the elongate member 614. The second magnet 604, although external to the user or implanted at a second location (the implantable device being at a first location), is placed operationally proximal to the first magnet 606. By doing so, the magnetic pole orientation of the second magnet 604 influences the magnetic pole direction of the first magnet 606. Put another way, a magnetic gear train may be created between the second magnet 604 and the first magnet 606, such that when the control device 602 rotates the second magnet 604, the first magnet 606 is rotated is an opposing direction (much like two mated gears). Rotating the first magnet 606 induces movement in the actuation device 9608, which moves the elongate member 614 to a restricted or unrestricted blood flow position or one or more intermediate positions. In embodiments with an actuation device 608 being a linear actuator connected to elongate member, the rotation of the first magnet 606 actuates the linear actuator (e.g., lead screw) to collapse or extend the elongate member 614. In some embodiments, both the first magnet 606 and the second magnet 604 may be permanent magnets. In further embodiments, the first magnet 606 is a permanent magnet and the second magnet 604 is an electromagnet.

As also described elsewhere herein, the control device 602 may receive signals from one or more optional sensors 610. These signals may be indicative of characteristics of total blood volume in the vasculature or the blood vessel in which the device is positioned. For example, when the control device 602 receives a signal indicative of a measured pressure higher than a predefined pressure threshold, the control device 602 can cause the second magnet 604 to rotate. The rotation of the second magnet 604 causes the first magnet 606 to rotate, which actuates the actuation device 608 to move the elongate member 614 towards a restricted blood flow position (e.g., a collapsed configuration or one or more intermediate configurations). Further, when the control device 602 receives a signal indicative of a measured pressure lower than a predefined pressure threshold, the control device 602 may cause the second magnet 604 to rotate in an opposing direction. The rotation of the second magnet 604 causes the first magnet 606 to rotate, thereby actuating the actuation device 608 to move the elongate member 614 towards the unrestricted blood flow position (e.g., an expanded configuration or one or more intermediate configurations). Sensor signals from sensor 610 may be transmitted to control device 602 via a wired connection or wirelessly. The described system is capable of positioning the elongate member in any position on or between the restricted position and unrestricted position. Additionally, and optionally, the system may include the capability of transmitting data to an optional remote computing device 612. The transmitted data may include sensor 610 measurements, valve position, actuation events, or any other data from the system.

FIG. 8B is an embodiment of a system 600B for modulating blood flow through a blood vessel. Components common to FIGS. 8A-8B share same element numbers and are described above with respect to FIG. 8A. Similar to FIG. 8A, FIG. 8B shows a magnetic gear train for manipulation of a valve of an implanted device. The control device 602 and second magnet 604, as in FIG. 3A, may be located external to the user or implanted (e.g., subcutaneously, intravascularly, etc.). Similar to FIG. 8A, the first magnet 606, actuation device 608, elongate member 614, and optional sensor 610 may be implanted within the user. Unlike FIG. 8A, this embodiment uses an implanted (in some embodiments, implanted subcutaneously) repeater magnet 605. This repeater magnet 605 may be used to extend the operational distance between the second magnet 604 and the first magnet 606, as it is implanted at an appropriate position between the two. Additionally, the repeater magnet 605 may be used to increase the torsional force that can be applied by the magnetic gear train. Further contemplated embodiments include a repeater module with a second power source operatively coupled to the repeater magnet 605 and capable of powering the rotation of the repeater magnet 605. Further, the rotation direction of the second magnet 604 and first magnet 606 are now the same, not opposing one another as in the previous embodiment. When manipulating the elongate member 614 towards the restricted or unrestricted blood flow positions, the second magnet 604 can be rotated in the same direction as the desired direction of the first magnet 606.

FIG. 11 illustrates another implantable device 250 for modulating flow through a vessel. The implantable device 250 may be self-expanding for ease of implantation, control, or manipulation. The device 250 includes a coiled wire 252 and a jacket 254 encasing at least a portion of the wire 252. The coiled wire 252 includes a first end 256 and a second end 258. The first end 256 may be coupled to or formed from a support frame (e.g., a stent) and the second end 258 may be free of attachment. The coiled wire 252 may be composed of or comprise a material with elastic and/or shape memory qualities. For example, the coiled wire 252 may be composed of or comprise a material or combination of materials, including, Nitinol®, other alloys (e.g., stainless steel, titanium, etc.), polymers (e.g., polyurethane, polycarbonate, polyacetal, etc.), or any other suitable materials with appropriate elastic and/or shape memory qualities. For example, if composed of or comprising Nitinol®, the implantable device 250 may be deformed into a smaller shape, for example a crimped shape, for delivery and may return to the illustrated shape when exposed to a predetermined temperature (e.g., body temperature) or when released from the confines of a delivery system, pusher tube, or catheter. Additionally, it may be advantageous to select a material or materials for the construction of the coiled wire 252 with bio-inert qualities, as to avoid eliciting a biological response or causing thrombosis when in contact with living tissues and/or biological fluids. The jacket 254 may include a composition of material or may comprise a material with scaling capabilities. Appropriate materials, alone or in combination, for the construction of the jacket 254 may include silicone, thermoplastic polyurethane, thermoplastic olefin, ethylene propylene diene monomer, butyl rubber, biocompatible cloth, tissues (e.g., synthetic polymers, porcine tissues, or natural biomaterials like collagen or hyaluronic acid, combined with cells to create artificial tissues), or any other suitable materials.

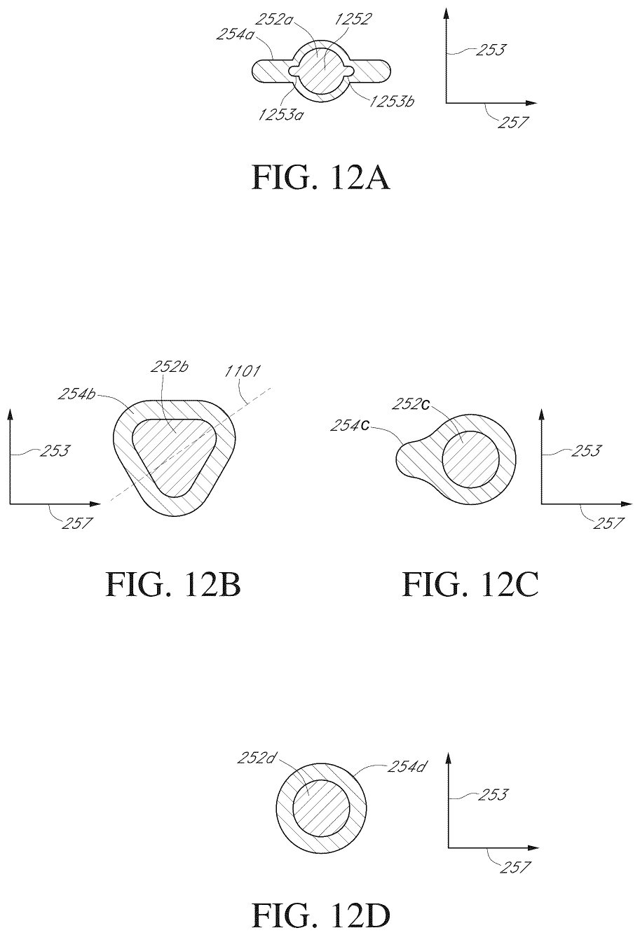

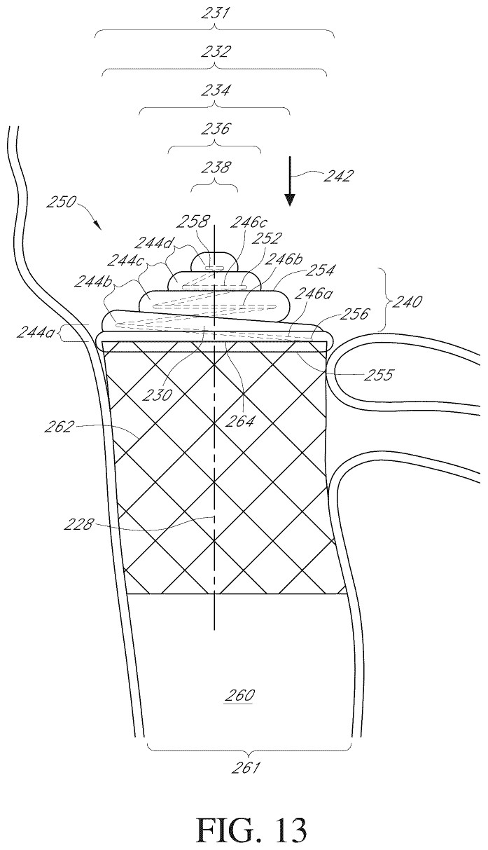

FIGS. 12A-12D illustrate embodiments of wire 252 and jacket 254 combinations from a cross-sectional perspective (cross-section along line A-A of the flexible member of FIG. 11). FIG. 12A illustrates a cross-sectional view of a coiled wire 252a and jacket 254a. The coiled wire 252a includes a substantially circular body 1252 with two protrusions 1253a-b on opposing sides of the circular body 1252. As such, the moment of inertia (i.e., area moment of inertia or second moment of inertia) for this cross-section may be greater with regard to an x-axis 257 than with regard to a y-axis 253. The imbalance of inertia may be advantageous in directing deformation caused by pressure or force applied to the coiled wire 252 (shown in FIG. 11). Said another way, the coiled wire 252a may be more prone to deflection along the y-axis 253. The jacket 254a cross-section may follow the contour of the coiled wire 252a cross-section. When forming a coil stack (as described for FIG. 13), the cross-sectional shape of the jacket 254a may be complementary to itself when conically stacked, and, thus, generates a scal when the device 250 is in a closed configuration. FIG. 12B illustrates a cross-sectional view of another embodiment of a coiled wire 252b and jacket 254b. The coiled wire 252b cross-section may include a non-uniform moment of inertia due to the triangular shape of the wire 252b cross-section and as such may include one or more axes of reduced inertia, for example, axis 1101. Axis 1101 may be aligned such that the bending of the coiled wire 252 (shown in FIG. 11) occurs about the reduced moment of inertia as measured by axis 1101. Orienting the coiled wire 252 (shown in FIG. 11) such that a reduced moment of inertia (relative to other moment orientations) of the wire cross-section is aligned with a pre-determined bend direction, and, thus, may increase the stability and reliability of the coiled wire 252b in forming a coil stack. The wire 252b cross-section may include greater resistance (i.e., greater moment of inertia) in bending in other directions than the pre-determined bend direction. FIG. 12C illustrates a cross-sectional view of another embodiment of a coiled wire 252c and jacket 254c. The cross-section of the coiled wire 252c includes a uniform moment of inertia (equal for any orientation, e.g., circular) and a non-uniform jacket 254c cross-section shape. When forming a coil stack (as described for FIG. 13), the cross-sectional shape of the jacket 254c may be complementary to itself when conically stacked, and, thus, generates a seal when the device 250 is in a closed configuration. FIG. 12D illustrates a cross-sectional view of another embodiment of a coiled wire 252d and jacket 254d. The cross-section of the coiled wire 252d and the jacket 254d are uniform in shape and with regard to moment of inertia.

FIG. 13 illustrates a side view of an implantable device 250 in a closed configuration. The illustrated state of the device 250 in FIG. 13 may be referred to as a natural state, collapsed configuration, a closed state, or neutral state. The implantable device 250 may further include a stent 262 (e.g., a support frame) to which the coiled wire 252 is coupled to (e.g., welded, woven, stitched, soldered, etc.), or integrally formed from (e.g., laser cut from hypotube). The implantable device 250 is illustrated as attached to one end of the stent 262, but it has been contemplated herein that attachment may be at any point along the stent 262. The stent 262 may include a lip 264. The lip 264 may include a coating 255 surrounding at least a portion of the lip 264 composed of or comprising material described herein for the jacket 254 (e.g., silicone, thermoplastic polyurethane, thermoplastic olefin, ethylene propylene diene monomer, butyl rubber, biocompatible cloth, tissues (e.g., synthetic polymers, or natural biomaterials like collagen or hyaluronic acid, porcine tissue, combined with cells to create artificial tissues), or any other suitable materials). The coiled wire 252 may include the wire 252 wound in a helical pattern or configuration around a conical (i.e., tapered) shaped core 230. Although a helical and conical formed coiled wire 252 is shown, other coiled wire 252 configurations are contemplated. For example, the helical winding of the coiled wire 252 may instead include non-circular coils, for example, elliptical coils, triangular coil, quadrilateral coils, pentagonal coils, hexagonal coils, heptagonal coils, octagonal coils, etc. Further, the core 230 may not be uniformly conical as shown with a straight taper. For example, the core 230 may include a concave taper or a convex taper. The coiled wire 252, when viewed from a side perspective (shown in FIG. 13), may form a plurality of coils, for example, a first coil 246a, a second coil 246b, and a third coil 246c, forming a coil stack narrowing in a direction parallel to longitudinal axis 228 of the stent 262 and away from the first end 256 of the coiled wire 252. Starting at the first end 256, the coiled wire 252 forms the first coil 246a with a diameter 232 greater than the diameter 234 of the second coil 246b, forms the second coil 246b with a diameter 234 greater than the diameter 236 of the third coil 246c, forms the third coil 246c, and ends at the second end 258. The diameter 232 of the first coil 246a may be greater than or approximately equal to the diameter 231 of the stent lip 264. The differences between diameter 231 and diameter 232, between diameter 232 and diameter 234, between diameter 234 and diameter 236, and between diameter 236 and the diameter 238 of the jacket 254 at or near the second end 258, are not great enough to allow the wire 252 and jacket 254 to collapse past the preceding coil or stent lip 264. Said another way, if a force (e.g., pressure causing flow) in the direction of arrow 242 were applied to the device 250, the wire 252 and jacket 254 portions near (i.e., portions between the third coil 246c and the second end 258) and at the second end 258 would contact the third coil 246c, the wire 252 and jacket 254 portions of the third coil 246c would contact the second coil 246b, the wire 252 and jacket 254 portions of the second coil 246b would contact the first coil 246a, and the wire 252 and jacket portions of the first coil 246a would contact the stent lip 264. As such, the compressible material of the jacket 254 and the coating 255 can create a seal between at least some portions of the device 250 and the stent lip 264. The seal created may be in response to, and may reduce or cease (e.g., prevent), flow created by pressure in a direction of arrow 242. In a static state, and without blood flow forces applied (excluding gravitational forces), the coiled wire 252 includes a dimension 244a between the wire 252 of the first coil 246a and the stent lip 264, a dimension 244b between the wire 252 of the first coil 246a and the wire 252 of the second coil 246b, a dimension 244c between the wire 252 of the second coil 246b and the wire 252 of the third coil 246c, and dimension 244d between the third coil 246c and the wire 252 near (i.e., portions between the third coil 246c and the second end 258) and at the second end 258. The coiled wire 252 may be formed in such a way that the memorized shape of the coiled wire 252 includes a dimension between the wire 252 portion of the first coil 246a and the stent lip 264 less than dimension 244a, a dimension between the wire 252 portion of the first coil 246a and the wire 252 portion of the second coil 246b less than dimension 244b, a dimension between the wire 252 portion of the second coil 246b and the wire 252 portion of the third coil 246c less than dimension 244c, and dimension between the wire 252 portion of the third coil 246c and the wire 252 portion near and/or at the second end 258 less than dimension 244d. With a coiled wire 252 formed with the aforementioned shape-memory dimensions, dimensions 244a, 244b, 244c, 244d are held by compression of the jacket 254 portions and coating 255 portions within each dimension 244a, 244b, 244c, 244d. Said another way, the coiled wire 252 compresses the jacket 254 portions and coating 255 portions between each portion (e.g., between the first coil 246a and the stent lip 264, between the first coil 246a and the second coil 246b, between the second coil 246b and the third coil 246c, between the third coil 246c and the second end 258 wire 252 portion) defining each dimension 244a, 244b, 244c, 244d and creating a compression seal. A device 250 configured as such will be normally closed unless acted upon by force and/or pressure. Alternatively, or additionally, the first coil 246a may form a seal with the wall of the vessel 260 in which the device 250 is placed. For example, the first coil 246a may include shape memory properties with a diameter greater than or equal to the inner diameter 261 of the vessel 260 in which the device 250 is positioned. Thus, a seal between the jacket 254 of the first coil 246a and the wall of the vessel 260 may be formed.

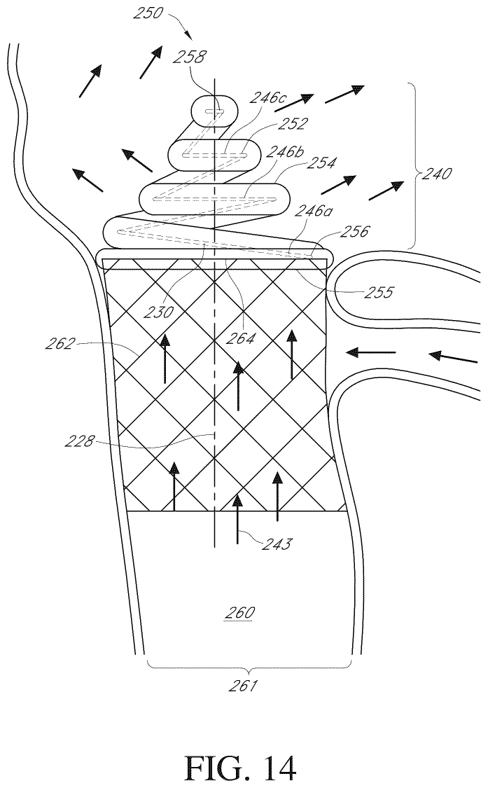

FIG. 14 illustrates a side view of the implantable device 250 in an open configuration or extended configuration. The implantable device 250 may be opened by blood flow and/or pressure in the direction of arrow 243. The amount of pressure to open the implantable device 250 may be pressures experienced under normal blood pressure ranges (e.g., from about 1 mmHg to about 15 mmHg, or from about 2 mmHg to about 8 mmHg, or from about 5 mmHg to about 15 mmHg). As previously described, the pressure acting in the direction of arrow 242 (shown in FIG. 13) creates a force upon the coiled wire 252 that is supported by the coil stack of the device 250 and creates a seal. In contrast, pressure acting on the implantable device 250 in the direction of arrow 243 creates a force on the coiled wire 252 that is unsupported, unlike the force created by pressure acting in the direction of arrow 242 (shown in FIG. 13) which is supported by the coil stack. As such, the force created by pressure in the direction of arrow 243 deforms (i.e., flexes) the coiled wire 252, expanding the coiled wire 252 in the direction of arrow 243 along the longitudinal axis 228 of the stent 262 and away from the first end 256 of the coiled wire 252. The deformation of the coiled wire 252 increases the coil stack dimension 240 and creates space for fluid to pass through. It may be advantageous to configure the coiled wire 252 to remain within the modulus of elasticity of the material of which the wire 252 is composed. Remaining within the modulus of elasticity may reduce cyclical stress and ensure the return of the coiled wire 252 to the closed state (i.e., return to the shape memory configuration). The amount of deformation, for example for a predefined flow rate through the device, may be adjusted by the number of coils forming the coiled wire 252. For example, an implantable device with more coils may experience a greater flow path area than an implantable device with less coils experiencing the same amount of wire deformation (i.e., flex). The flow path area is the area between the coils (e.g., coils 246a, 246b, 246c), the stent lip 264, and the second end 258 portion, thus embodiments with more coils include greater flow path areas. The implantable device 250 is illustrated with three coils 246a, 246b, 246c, but more and less coils are contemplated (e.g., one coil, two coils, four coils, five coils, six coils, etc.). The device 250 may include a control element 502 as described for FIG. 5. The control element 502 may be adapted to maintain the coiled wire in one or more of: the collapsed configuration, the extended configuration, or the one or more intermediate positions. In some embodiments, the control element 502 is adapted to reversibly maintain the coiled wire in one or more of: the collapsed configuration, the extended configuration, or the one or more intermediate positions. In some embodiments, the control element 502 further includes a linear actuator 504 configured to tension the control element to maintain the coiled wire in one or more of: the collapsed configuration, the extended configuration, or the one or more intermediate positions.

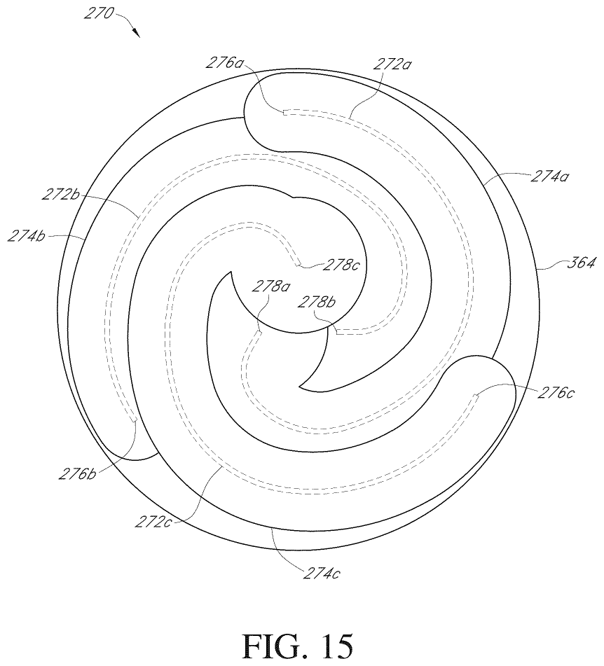

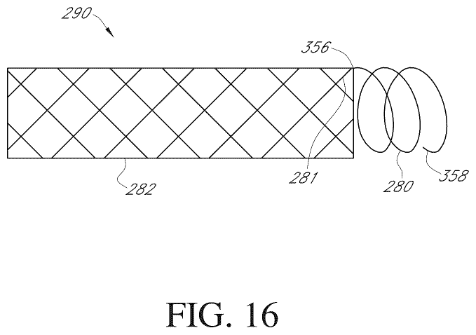

FIG. 15 illustrates another embodiment of an implantable device 270 for the modulating blood flow. The device 270 includes a plurality of coiled wires. For example, device 270 may include a first coiled wire 272a, a second coiled wire 272b, and a third coiled wire 272c. The implantable device 270 may include a support frame (e.g., a stent 262 shown in FIG. 14) to which each coiled wire 272a, 272b, 272c is coupled. The first coiled wire 272a, the second coiled wire 272b, and the third coiled wire 272c may be coupled to (e.g., welded, woven, stitched, soldered, etc.), or integrally formed from (e.g., frame and wire laser cut from a hypotube, frame and wire water jet cut from a hypotube), the support frame at a first wire fixed end 276a, a second wire fixed end 276b, and a third wire fixed end 276c, respectively. The first coiled wire 272a, the second coiled wire 272b, and the third coiled wire 272c may end at a first wire terminus 278a, a second wire terminus 278b, and a third wire terminus 278c, respectively. The first wire terminus 278a, the second wire terminus 278b, and the third wire terminus 278c may be free (i.e., uncoupled as described for the second end 258 in FIGS. 11, 13 and 14). The first coiled wire 272a, the second coiled wire 272b and the third coiled wire 272c may include a first jacket 274a, a second jacket 274b and a third jacket 274c, respectively. Jackets 274a, 274b, 274c may be composed of or comprise materials, or a combination of materials, appropriate for creating a scal, for example, silicone, thermoplastic polyurethane, thermoplastic olefin, ethylene propylene diene monomer, butyl rubber, biocompatible cloth, porcine tissue, tissues (e.g., synthetic polymers, or natural biomaterials like collagen or hyaluronic acid, combined with cells to create artificial tissues), or any other suitable materials. The device 270 forms a coil stack as described for implantable device 250 (shown in FIGS. 11, 13 and 14), including a coil stack dimension 240 (shown in FIGS. 13 and 14) and with the coil stack narrowing in a direction parallel to longitudinal axis 228 of the stent 262 and away from the first end 256 of the coiled wire 252 (as described in FIG. 13), but is formed by the three approximately parallel coiled wires 272a, 272b, 272c. Approximately parallel coiled wires 272a, 272b, 272c may be formed such that the coiled wires are aligned in the same direction, about the same axis (longitudinal axis 228 shown in FIGS. 13 and 14), and include the same pitch at the same points along the coiled wires 272a, 272b, 272c. In a closed configuration, as shown in FIG. 15, the first coiled wire 272a is supported by, and thereby creates a seal with, the lip 364 of a support frame (e.g., the stent 262 shown in FIGS. 13 and 14) and the third coiled wire 272c. Further, the second coiled wire 272b is supported by, and thereby creates a seal with, the lip 364 of a support frame (e.g., the stent 262 shown in FIGS. 13 and 14) and the first coiled wire 272a. Further, the third coiled wire 272a is supported by, and thereby creates a scal with, the lip 364 of a support frame (e.g., the stent 262 shown in FIGS. 13 and 14) and the second coiled wire 272b. The device 270 may include portions of the first jacket 274a, the second jacket 274b, and the third jacket 274c that are complementary to one another and/or the lip 364 of the support frame of the device 270. For example, the portion of jacket 274a surrounding the first wire terminus 278a, the portion of jacket 274b surrounding the second wire terminus 278b, and the portion of jacket 274c surrounding the third wire terminus 278c may be complementary to one another such that a seal is formed at the point at which the three termini 278a, 278b, 278c meet. As such, the described coil stack of the device 270 can create a seal when experiencing a pressure or force parallel to the longitudinal axis 228 (shown in FIGS. 13 and 14) of the support frame and in a direction from the termini 278a, 278b, 278c to the fixed ends 276a, 276b, 276c (i.e., in the direction of arrow 242). If the device 270 experiences a pressure or force parallel to the longitudinal axis 228 (shown in FIGS. 13 and 14) of the support frame and in a direction from the fixed ends 276a, 276b, 276c to the termini 278a, 278b, 278c (i.e., in the direction of arrow 243), the wires 272a, 272b, 272c may deform (i.e., flex) outwards along the longitudinal axis 228 (shown in FIG. 14) and create a flow path for fluid. The multi-coiled wire device 270 functions to allow fluid flow in a first direction (i.e., in the direction of arrow 243 along the longitudinal axis 228, shown in FIGS. 13 and 14) of the support frame and in a direction from the fixed ends 276a, 276b, 276c to the termini 278a, 278b, 278c of a coiled wires 272a, 272b, 272c) and seal against a fluid flow in a second direction (i.e., in the direction of arrow 242 along the longitudinal axis 228, shown in FIG. 13) of the support frame and in a direction from the termini 278a, 278b, 278c to the fixed ends 276a, 276b, 276c of coiled wires 272a, 272b, 272c) as the device 250 described in FIGS. 13 and 14. In contrast, and advantageously, the deformation forces that may be experienced by the one coiled wire 252 of the device 250 described in FIGS. 13 and 14, may be equalized across the three coiled wires 272a, 272b, 272c of device 270. Equalizing the deformation forces across the three coiled wires 272a, 272b, 272c may reduce the stress experienced by each wire 272a, 272b, 272c. The reduction of stress may increase the cyclical lifespan of the device 270. The device 270 is illustrated with three coiled wires 272a, 272b, 272c, but embodiments with more and less coiled wires have been contemplated herein (e.g., two coiled wires, four coiled wires, five coiled wires, six coiled wires, etc.). Additionally, or alternatively, the portions of the first coiled wire 272a, the second coiled wire 272b, and the third coiled wire 272c which contact the lip 364 of the support frame may contact the inner diameter 261 of the vessel 260 (shown in FIGS. 13 and 14) in which the device 270 is located. As such, a seal may be created between the aforementioned wire 272a, 272b, 272c portions and the wall of the vessel 260.

Implantable devices with coiled wires may be designed for multiple implant locations and actuation pressure ranges. Actuation pressure ranges may be specific to the gauge of wire used in the coiled wire. For example, the diameter of wire used may be in a range of about 0.25 mm to about 3.00 mm; about 0.50 mm to about 2.00 mm; etc. The selected diameter of wire may be based upon several factors, including, the overall size of the implantable device or the diameter of the orifice within the implantable device may be placed. Further, implantable device with coiled wires may be manipulated into a smaller and/or alternate form during implantation, thus it may be advantageous for these devices to self-expand, for example due to shape memory properties, into a functional shape.

FIG. 16 illustrates a side view of an implantable device 290. The implantable device 290 includes a support frame 282 (e.g., a stent) and a coiled wire 280. The implantable device 290 and the support frame 282 may both be manipulated into a smaller and/or alternate form during implantation, as such the support frame 282 and coiled wire 280 may self-expand, for example due to shape memory properties, to the functional shape shown in FIG. 16. The coiled wire 280 includes a first end 356 and a second end 358. The coiled wire 280 may be integrally formed from the support frame 282 as shown. For example, both the frame and the coiled wire(s) may be laser cut from a hypotube. For example, and as shown, a wire 281 may be used to form at least a portion of the support frame 282 and form the coiled wire 280, beginning at the first end 356 and ending at the second end 358.

### Methods

The methods described herein can provide blood flow occlusion therapy and may relate to venous occlusion therapy using implantable and/or electronically controlled flow restricting devices for the treatment of acute heart failure. Some devices of the methods may be non-implantable or partially implantable. In some examples, the devices of the methods described herein generally function to occlude or partially occlude a blood vessel, such as the SVC or the IVC. In some examples, the methods described herein have been contemplated for use in a patient/user having chronic heart failure and/or chronic kidney disease but may be used in any vessel needing flow regulation therethrough.

As shown in FIG. 9, a method 700 for modulating blood flow through a blood vessel of one embodiment includes providing an implantable device for modulating blood flow in block S702; implanting the device in a blood vessel in block S704; optionally detecting a blood pressure with a sensor in optional block S706; optionally categorizing the detected blood pressure into a first, second, or third category in optional block S708; and optionally adjusting an actuation device of the implantable device in optional block S710.

In block S702, the method 700 includes providing an implantable device for modulating blood flow. In some embodiments, the device includes a support frame and a spiral-shaped flow occlusion strip adapted to flex between a collapsed configuration and an extended configuration. As described above, the flow occlusion strip has a first strip end and a second strip end. The first strip end is coupled to the support frame and the second strip end is extendable and collapsible relative to the support frame. In some embodiments, the flow occlusion strip is adapted to flex to one or more of: a collapsed configuration, an extended configuration, or one or more intermediate positions, according to a blood pressure in the blood vessel.

Alternatively, in block 702, the device includes an expandable cage and one or more leaflets extending between a first end and a second end of the expandable cage. The cage is manipulatable between an expanded configuration in which the one or more leaflets permit blood flow through the cage and a collapsed configuration in which the one or more leaflets prolapse and at least partially occlude the vessel in which the device is implanted.

In some embodiments, the implantable device further includes a control element adapted to reversibly maintain the flow occlusion strip or cage in one or more of: a collapsed configuration, an extended or expanded configuration, or one or more intermediate positions. In further embodiments, the control element includes a linear actuator configured to tension the control element to maintain the flow occlusion strip or cage in one or more of: a collapsed configuration, an expanded or extended configuration, or one or more intermediate positions. In additional embodiments, the linear actuator includes a material having shape memory and/or pseudo elastic properties, as described herein. In still further embodiments, the linear actuator includes Nitinol®. A linear actuator may also be a spring, piston, hydraulic, electromechanical actuator, etc., as described above.

In block S704, the method 700 includes implanting the device in a blood vessel. In some embodiments, the blood vessel is at least a portion of a superior vena cava or an inferior vena cava. Various delivery techniques can be employed as described herein.

In optional block S706, the method 700 optionally includes for detecting a blood pressure with an optional sensor. In these embodiments, the device (or a stent that the device is disposed within) can include optional sensor. The actuation device can include a linear actuator coupled to the control element, a power source, and a microcontroller electrically coupled to the power source, the linear actuator, and the sensor. As described herein, various sensors, including optical sensors, can be used to measure a blood pressure of the blood vessel. The sensor may or may not be physically coupled to the implantable device, in some embodiments.

In optional block S708, the method 700 optionally includes categorizing the detected blood pressure into a first, second, or third blood pressure state. In some embodiments, the first pressure state is when the detected blood pressure is, for example, about 30 mmHg or less, about 25 mmHg or less, or about 20 mmHg or less; the second pressure state is when the detected blood pressure is, for example, about 25 mmHg or higher or about 30 mmHg or higher; and the third pressure state is when the detected blood pressure is between about 10 mmHg and about 30 mmHg. In some embodiments, this categorization is performed by the microcontroller. In some embodiments, the categorization is performed by an optional remote computing device in communication with the microcontroller.

In optional block S710, the method 700 optionally includes adjusting the actuator device. In various embodiments, adjusting the actuator device includes one of: tensioning the control element, using the linear actuator, to maintain the flow occlusion strip or cage in the collapsed configuration in response to the first pressure state; releasing tension in the control element, using the linear actuator, to maintain the flow occlusion strip or cage in an extended or expanded configuration in response to the second pressure state; and tensioning the control element, using the linear actuator, to maintain the flow occlusion strip in one or more intermediate positions in response to the third pressure state. In some embodiments, the method 700 then returns to optional block S706 and repeats blocks S706-S710. In some embodiments, the method 700 repeats blocks S706-S710 indefinitely at a predetermined frequency (e.g., once per second or less).

FIG. 10 illustrates a schematic representation of portions of a subject 800. The flow modulating devices described herein (e.g., represented in FIGS. 1A-1E, 2A-2B, 3A-3B, 4-5, 6A-6B, 7A-7C, and 8A-8B) may be introduced (e.g., implanted) in vasculature of the body. In general, the device 802 may represent any of the flow modulating devices described herein (e.g., shown in FIGS. 1A-1E, 2A-2B, 3A-3B, 4-5, 6A-6B, 7A-7C, 8A-8B, 11 and 13-16) and may include the same or similar functionality and/or structures. In some examples, the device 802 may be implanted in or near to a portion of the superior vena cava (SVC) 804. In some examples, the device 802 may be implanted in or near to a portion of the inferior vena cava (IVC) 806. The subject 800 is illustrated with a representation of a portion of the vasculature system to generally illustrate the SVC 804 and the IVC 806 within the subject 800. However, it is to be understood that no dimensions or relative sizes of components may be inferred from the relative sizes and dimensions of elements in the figures.

The subject 800 includes a number of vessels and organs that may circulate blood throughout the body. For example, renal veins 808a and 808b drain blood from respective right kidney 810 and left kidney 812. Renal veins 808a and 808b connect to the IVC 806. Blood from the aorta 814 flows to the IVC 806. Blood travels from the aorta 814 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 806 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized.

For example, because blood flow from the splanchnic venous circulation is directed through hepatic veins and into the IVC 806, devices (as described herein) may be placed into the IVC 806 to limit blood flow to allow the splanchnic venous circulation to expand with increased blood volume. This may also allow the body to accumulate blood volume in the splanchnic venous circulation, which can maximize the downstream drop of pressure relative to upstream increase of pressure. Similarly, devices (as described herein) may be placed into the SVC 808 to limit blood flow to allow the reservoir to expand with increased blood volume. Furthermore, the flow modulating devices described herein may be placed in either the IVC 806 and/or SVC 808 to alleviate pressure in the right side of the atrium of the heart 816 and/or regulate renal venous pressure and kidney function. Another example positioning of a flow modulating device may be in the IVC below the renal veins. This positioning may have a similar effect as the SVC location, as it may allow the flow modulating device to maintain renal venous pressure, which can correlate with sustained renal function and diuresis.

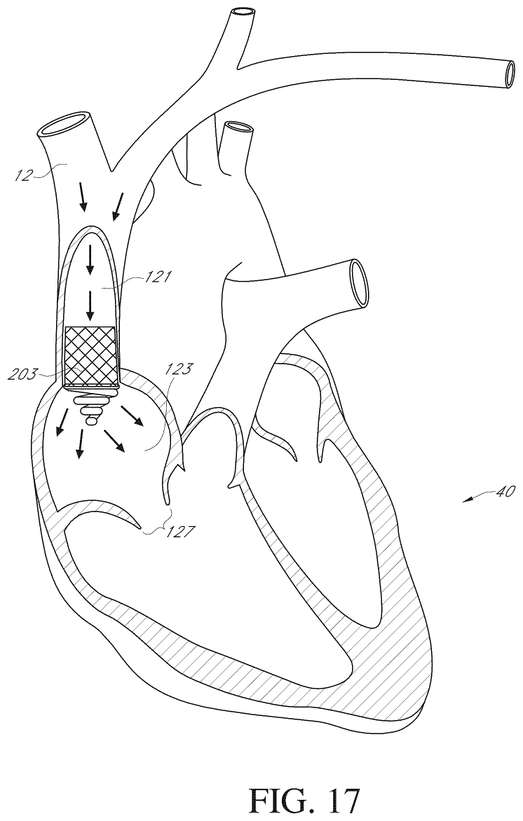

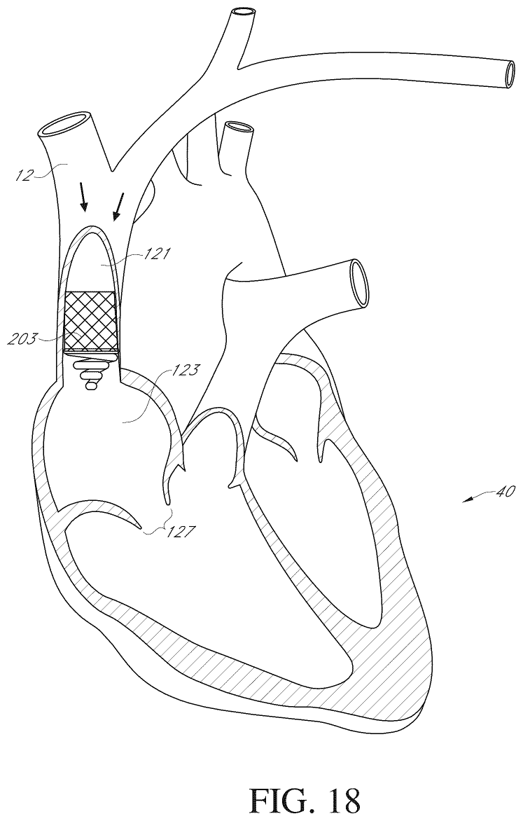

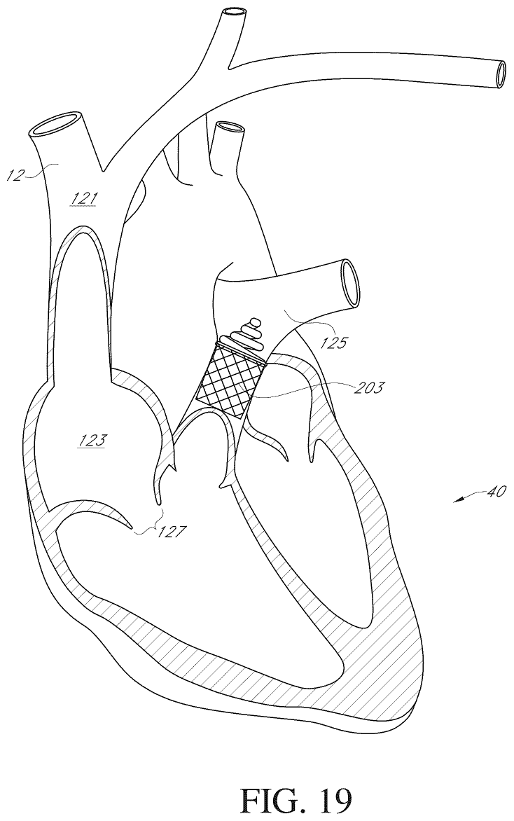

Patients may experience conditions in which blood flow undesirably reverses direction due to improper closure of a diseased valve. Examples of valve types include the various natural valves of the heart. For example, tricuspid valve regurgitation can cause the tricuspid valve of the heart 816 to improperly seal and allow blood to flow backwards into the right atrium. The flow modulating device 802 (representing the devices described herein) may be used as a method of treatment to treat tricuspid valve regurgitation. The flow modulating devices described herein may be placed in either the IVC 806 and/or SVC 808 to restrict blood flow to only the desired direction. Alternatively, the flow modulating devices described herein may be placed within a diseased valve, or in place of a diseased valve (i.e., replace the diseased valve). For example, the devices described in FIGS. 11 and 13-16 may be used to allow normal blood flow through the tricuspid valve, and close if the blood flow direction reverses. For example, and as shown in FIG. 17, a flow modulating device 203 (e.g., the device 250 of FIGS. 11, 13, and 14, the device 270 of FIG. 15, the device 290 of FIG. 16) may be implanted in the superior vena cava 121 with a portion of the device 203 in the right atrium 123, at least during certain periods of actuation (e.g., while open). For example, and as shown in FIG. 18, a flow modulating device 203 (e.g., the device 250 of FIGS. 11, 13, and 14, the device 270 of FIG. 15, the device 290 of FIG. 16) may be implanted completely within the superior vena cava 121. For example, and as shown in FIG. 19, a flow modulating device 203 (e.g., the device 250 of FIGS. 11, 13, and 14, the device 270 of FIG. 15, the device 290 of FIG. 16) may be implanted within the pulmonary artery 125. With a flow modulating device 203 (e.g., the device 250 of FIGS. 11, 13, and 14, the device 270 of FIG. 15, the device 290 of FIG. 16) implanted as shown and described for FIGS. 17-19, if the tricuspid valve 127 improperly seals (e.g., during tricuspid valve regurgitation) blood may not be allowed to flow backwards into the right atrium 123, or may be, at least, limited in the amount of blood volume which is allowed to flow backwards into the right atrium 123.

In some examples, the flow modulating device 802 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 802 may be used as a method of treatment to regulate pressure in the right atrium of the heart or regulate pressure in any bodily vessel. Further, the flow modulating device 802 may be used as a method of treatment to improve function of the kidneys in patients having reduced kidney function due to pressure in the venous system.

For example, any of the implantable devices and/or systems described herein may be configured to modulate a volume of blood flowing from a superior vena cava into a right atrium to decrease right atrial pressure.

For example, any of the implantable devices and/or systems described herein may be configured to modulate a volume of blood flowing from a first vessel into a second vessel to decrease pressure in the second vessel.

Further for example, any of the implantable devices and/or systems described herein may be used to perform a method including restricting blood flow within a blood vessel.

Still further for example, any of the implantable devices and/or systems described herein may be used to perform a method of treatment for a subject having one or both of: congestive heart failure or chronic kidney disease. The method may include restricting blood flow within the blood vessel.

The spatially relative terms “outer,” “inner,” “upper,” “lower,” “below,” “above,” “vertical,” “horizontal,” and similar terms, may be used herein for case of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the implantable device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### EXAMPLES

### Example 1

An implantable device for modulating blood flow through a blood vessel, the implantable device comprising: a support frame; a coiled wire forming a coil stack, wherein the coiled wire is adapted to flex between a collapsed configuration and an extended configuration, wherein the coiled wire has a first end and a second end, and wherein the first end is coupled to or formed from the support frame and the second end is extendable and collapsible relative to the support frame; and a jacket surrounding at least a portion of the coiled wire.

### Example 2

The implantable device of any one of the preceding examples, but particularly Example 1, wherein the coiled wire is conical and wherein the formed coil stack is conical.

### Example 3

The implantable device of any one of the preceding examples, but particularly Example 1, wherein the coiled wire comprises a cross-section that includes a non-uniform moment of inertia.

### Example 4

The implantable device of any one of the preceding examples, but particularly Example 1, wherein the coiled wire comprises a cross-section that includes a uniform moment of inertia.

### Example 5

The implantable device of any one of the preceding examples, but particularly Example 1, wherein the coiled wire is adapted to flex to one or more intermediate positions between the collapsed configuration and the extended configuration.

### Example 6

The implantable device of any one of the preceding examples, but particularly Example 5, wherein, when the coiled wire is at least partially flexed, the second end is configured to extend away from the first end along a substantially longitudinal axis of the support frame in a same direction as a blood flow direction through the blood vessel.

### Example 7

The implantable device of any one of the preceding examples, but particularly Example 5, wherein the coiled wire is adapted to flex to one or more of: the collapsed configuration, the extended configuration, or the one or more intermediate positions, in response to a blood pressure acting on the device.

### Example 8

The implantable device of any one of the preceding examples, but particularly Example 5, further comprising a control element coupled to the coiled wire and adapted to move the coiled wire between one or more of: the collapsed configuration, the extended configuration, or the one or more intermediate positions.

### Example 9

The implantable device of any one of the preceding examples, but particularly Example 8, wherein the control element comprises a linear actuator configured to tension the control element to move the coiled wire to one or more of: the collapsed configuration, the extended configuration, or the one or more intermediate positions.

### Example 10

The implantable device of any one of the preceding examples, but particularly Example 8, further comprising: a sensor; and an actuation device comprising: a linear actuator coupled to the control element, a power source, and a microcontroller electrically coupled to the power source, the linear actuator, and the sensor, wherein the linear actuator is configured to tension the control element to maintain the coiled wire in the collapsed configuration in response to a first pressure state sensed by the sensor, wherein the linear actuator is configured to release tension in the control element to maintain the coiled wire in the extended configuration in response to a second pressure state sensed by the sensor, and wherein the linear actuator is configured to tension the control element to maintain the coiled wire in the one or more intermediate positions during a range of pressure states sensed by the sensor between the first and second pressure state.

### Example 11

The implantable device of any one of the preceding examples, but particularly Example 1, wherein the coiled wire comprises a material with shape memory properties.

### Example 12

The implantable device of any one of the preceding examples, but particularly Example 11, wherein the shape memory of the coiled wire is a shape of the coiled wire in the collapsed configuration.

### Example 13

The implantable device of any one of the preceding examples, but particularly Example 1, wherein the jacket comprises a material, or a combination of materials, appropriate for creating a seal.

### Example 14

The implantable device of any one of the preceding examples, but particularly Example 13, wherein materials suitable for creating a seal comprising silicone, thermoplastic polyurethane, thermoplastic olefin, ethylene propylene diene monomer, butyl rubber, biocompatible cloth, synthetic polymer tissue, porcine tissue, collagen, and hyaluronic acid.

### Example 15

The implantable device of any one of the preceding examples, but particularly Example 13, wherein, in the collapsed configuration, the jacket forms a seal between contacting portions.

### Example 16

The implantable device of any one of the preceding examples, but particularly Example 15, wherein, in the collapsed configuration, the jacket comprises a cross-section complementary between the contacting portions.

### Example 17

A method of modulating blood flow within a blood vessel, comprising using the device of any one of examples 1-16 to allow blood flow in a first direction within the blood vessel and prevent blood flow in a second direction within the blood vessel.

### Example 18

A method of treating tricuspid valve regurgitation, comprising using the device of any one of the examples 1-16 to allow blood flow in a first direction within at least one of: a superior vena cava, an inferior vena cava, or a pulmonary artery, and prevent blood flow in a second direction within at least one of: the superior vena cava, the inferior vena cava, or the pulmonary artery.

### Example 19

A method of treating blood flow reversal through a diseased valve, comprising using the device of any one of the examples 1-16 to allow blood flow in a first direction within a blood vessel connecting to the valve and prevent blood flow in a second direction within a blood vessel connecting to the valve.

### Example 20

A method for modulating blood flow through a blood vessel, the method comprising: providing an implantable device for modulating blood flow, the implantable device comprising: a support frame, and one or more coiled wires adapted to flex between a collapsed configuration to at least partially occlude the blood vessel and an extended configuration to at least partially open the blood vessel, wherein the one or more coiled wires each include a first end and a second end, and wherein the first end is coupled to the support frame and the second end is extendable and collapsible relative to the support frame; and implanting the device in the blood vessel.

### Example 21

The method of any one of the preceding examples, but particularly Example 20, wherein the one or more coiled wires are conical.

### Example 22

The method of any one of the preceding examples, but particularly Example 20, wherein the one or more coiled wires each include a respective jacket surrounding at least a portion of each respective coiled wire.

### Example 23

The method of any one of the preceding examples, but particularly Example 20, wherein the one or more coiled wires are adapted to flex to one or more of: the collapsed configuration, the extended configuration, or one or more intermediate positions, according to a blood pressure in the blood vessel.

### Example 24

The method of any one of the preceding examples, but particularly Example 20, wherein the device further comprises a control element adapted to reversibly maintain the one or more coiled wire in one or more of: the collapsed configuration, the extended configuration, or one or more intermediate positions.

### Example 25

The method of any one of the preceding examples, but particularly Example 24, wherein the control element comprises a linear actuator configured to tension the control element to maintain the one or more coiled wire in one or more of: the collapsed configuration, the extended configuration, or the one or more intermediate positions.

### Example 26

The method of any one of the preceding examples, but particularly Example 20, wherein the blood vessel is a superior vena cava, pulmonary artery, or an inferior vena cava.

### Example 27

The method of any one of the preceding examples, but particularly Example 20, wherein the implantable device further comprises: a sensor; and an actuation device comprising: a linear actuator coupled to a control element, a power source, and a microcontroller electrically coupled to the power source, the linear actuator, and the sensor, and wherein the method further comprises: detecting a blood pressure with the sensor; categorizing the detected blood pressure as within a predetermined first pressure state, a predetermined second pressure state, or a predetermined third pressure state that is between the first and second pressure states, wherein the categorizing is performed by the microcontroller; and adjusting the actuation device wherein adjusting the actuation device is one of: activating the linear actuator to tension the control element to maintain the one or more coiled wire in the collapsed configuration in response to the first pressure state; activating the linear actuator to release tension in the control element to maintain the one or more coiled wire in the extended configuration in response to the second pressure state; and activating the linear actuator to tension the control element to maintain the one or more coiled wire in one or more intermediate positions in response to the third pressure state.

### Example 28

The method of any one of examples 20-27, further comprising modulating blood flow within the blood vessel.

### Example 29

An implantable device for modulating blood flow through a blood vessel, the implantable device comprising: a support frame; and a plurality of coiled wires forming a coil stack, wherein each coiled wire is adapted to flex between a collapsed configuration and an extended configuration, wherein each coiled wire has a fixed end and a terminus, and wherein the fixed end of each coiled wire is coupled to or formed from the support frame and the terminus of each coiled wire is extendable and collapsible relative to the support frame, and wherein each coiled wire includes a respective jacket surrounding at least a portion of each coiled wire.

### Example 30

The implantable device of any one of the preceding examples, but particularly Example 29, wherein the plurality of coiled wires comprises a cross-section that includes a non-uniform moment of inertia.

### Example 31

The implantable device of any one of the preceding examples, but particularly Example 29, wherein the plurality of coiled wires comprises a cross-section that includes a uniform moment of inertia.

### Example 32

The implantable device of any one of the preceding examples, but particularly Example 29, wherein, in the collapsed configuration, the respective jackets of the coiled wires are complementary to one another.

### Example 33

The implantable device of any one of the preceding examples, but particularly Example 29, wherein the plurality of coiled wires is conical and wherein the formed coil stack is conical.

### Example 34

The implantable device of any one of the preceding examples, but particularly Example 29, wherein the plurality of coiled wires is approximately parallel.

### Example 35

The implantable device of any one of the preceding examples, but particularly Example 29, wherein each coiled wire is adapted to flex to one or more intermediate positions between the collapsed configuration and the extended configuration.

### Example 36

The implantable device of any one of the preceding examples, but particularly Example 29, wherein, when each coiled wire is at least partially flexed, the terminus of each coiled wire is configured to extend away from the fixed end of each coiled wire along a substantially longitudinal axis of the support frame in a same direction as a blood flow direction through the blood vessel.

### Example 37

The implantable device of any one of the preceding examples, but particularly Example 29, wherein the plurality of coiled wires comprises a material with shape memory properties.

### Example 38

The implantable device of any one of the preceding examples, but particularly Example 37, wherein the shape memory of the plurality of coiled wires is a shape of the plurality of coiled wires in the collapsed configuration.

### Example 39

The implantable device of any one of the preceding examples, but particularly Example 29, wherein the respective of jackets of the coiled wires comprise a material, or a combination of materials, appropriate for creating a scal.

### Example 40

The implantable device of any one of the preceding examples, but particularly Example 39, wherein materials suitable for creating a scal comprising silicone, thermoplastic polyurethane, thermoplastic olefin, ethylene propylene diene monomer, butyl rubber, biocompatible cloth, synthetic polymer tissue, porcine tissue, collagen, and hyaluronic acid.

### Example 41

The implantable device of any one of the preceding examples, but particularly Example 39, wherein, in the collapsed configuration, the respective jackets of the coiled wires form a seal between one another.

### Example 42

A method of restricting blood flow within a blood vessel, comprising using the device of any one of the examples 22-41 to modulate blood flow within the blood vessel.

### Example 43

A method of treating tricuspid valve regurgitation, comprising using the device of any one of the examples 22-41 to allow blood flow in a first direction within at least one of: a superior vena cava, an inferior vena cava, or a pulmonary artery, and prevent blood flow in a second direction within at least one of: the superior vena cava, the inferior vena cava, or the pulmonary artery.

### Example 44

A method of treating blood flow reversal through a diseased valve, comprising using the device of any one of the examples 22-41 to allow blood flow in a first direction within a blood vessel connecting to the valve and prevent blood flow in a second direction within a blood vessel connecting to the valve.

### Example 45

An implantable device for modulating blood flow through a blood vessel, the implantable device comprising: a support frame; and a coilable flow occlusion strip adapted to flex between a collapsed configuration and an extended configuration, wherein the coilable flow occlusion strip has a first end and a second end, and wherein the first end is coupled to the support frame and the second end is extendable and collapsible relative to the support frame.

### Example 46

The implantable device of any one of the preceding examples, but particularly Example 45, wherein the flow occlusion strip is adapted to flex to one or more intermediate positions between the collapsed configuration and the extended configuration.

### Example 47

The implantable device of any one of the preceding examples, but particularly Example 46, wherein, when the coilable flow occlusion strip is at least partially flexed, the second end is configured to extend away from the first end along a substantially longitudinal axis of the support frame in a same direction as a blood flow direction through the blood vessel.

### Example 48

The implantable device of any one of the preceding examples, but particularly Example 46, wherein, when the coilable flow occlusion strip is at least partially flexed, the second strip end is configured to extend away from the first end along a substantially longitudinal axis of the support frame in an opposite direction of a blood flow direction through the blood vessel.

### Example 49

The implantable device of any one of the preceding examples, but particularly Example 46, wherein the coilable flow occlusion strip is adapted to flex to one or more of: the collapsed configuration, the extended configuration, or the one or more intermediate positions, in response to a blood pressure acting on the device.

### Example 50

The implantable device of any one of the preceding examples, but particularly Example 49, wherein the support frame further comprises a tensioner adapted to maintain the coilable flow occlusion strip in the collapsed configuration under a first range of blood pressures, maintain the coilable flow occlusion strip in the extended configuration under a second range of blood pressures, and maintain the coilable flow occlusion strip in one or more intermediate positions under a third range of blood pressures between the first and second ranges.

### Example 51

The implantable device of any one of the preceding examples, but particularly Example 46, further comprising a control element adapted to maintain the coilable flow occlusion strip in one or more of: the collapsed configuration, the extended configuration, or the one or more intermediate positions.

### Example 52

The implantable device of any one of the preceding examples, but particularly Example 51, wherein the control element is further adapted to reversibly maintain the coilable flow occlusion strip in one or more of: the collapsed configuration, the extended configuration, or the one or more intermediate positions.

### Example 53

The implantable device of any one of the preceding examples, but particularly Example 52, wherein the control element comprises a linear actuator configured to tension the control element to maintain the coilable flow occlusion strip in one or more of: the collapsed configuration, the extended configuration, or the one or more intermediate positions.

### Example 54

The implantable device of any one of the preceding examples, but particularly Example 53, wherein the control element comprises a material having shape memory or pseudo elastic properties.

### Example 55

The implantable device of any one of the preceding examples, but particularly Example 54, wherein the control element comprises shape memory alloy.

### Example 56

The implantable device of any one of the preceding examples, but particularly Example 55, wherein the linear actuator comprises a device configured to deliver current to the control element to elongate or shorten the control element.

### Example 57

The implantable device of any one of the preceding examples, but particularly Example 52, further comprising: a sensor; and an actuation device comprising: a linear actuator coupled to the control element, a power source, and a microcontroller electrically coupled to the power source, the linear actuator, and the sensor, wherein the linear actuator is configured to tension the control element to maintain the coilable flow occlusion strip in the collapsed configuration in response to a first pressure state sensed by the sensor, wherein the linear actuator is configured to release tension in the control element to maintain the coilable flow occlusion strip in the extended configuration in response to a second pressure state sensed by the sensor, and wherein the linear actuator is configured to tension the control element to maintain the coilable flow occlusion strip in the one or more intermediate positions during a range of pressure states sensed by the sensor between the first and second pressure state.

### Example 58

A method of restricting blood flow within a blood vessel, comprising using the device of any one of examples 45-54 to restrict blood flow within the blood vessel.

### Example 59

A method of treatment for a subject having one or both of: congestive heart failure or chronic kidney disease, comprising using the device of any one of examples 45-54 to restrict blood flow within the blood vessel.

### Example 60

A method for modulating blood flow through a blood vessel, the method comprising: providing an implantable device for modulating blood flow, the implantable device comprising: a support frame; and a coilable flow occlusion strip adapted to flex between a collapsed configuration to at least partially occlude the blood vessel and an extended configuration to at least partially open the blood vessel, wherein the flow occlusion strip has a first end and a second end, and wherein the first end is coupled to the support frame and the second end is extendable and collapsible relative to the support frame; and implanting the device in the blood vessel.

### Example 61

The method of any one of the preceding examples, but particularly Example 60, wherein the coilable flow occlusion strip is adapted to flex to one or more of: the collapsed configuration, the extended configuration, or one or more intermediate positions, according to a blood pressure in the blood vessel.

### Example 62

The method of any one of the preceding examples, but particularly Example 60, wherein the device further comprises a control element adapted to reversibly maintain the coilable flow occlusion strip in one or more of: the collapsed configuration, the extended configuration, or one or more intermediate positions.

### Example 63

The method of any one of the preceding examples, but particularly Example 62, wherein the control element comprises a linear actuator configured to tension the control element to maintain the coilable flow occlusion strip in one or more of: the collapsed configuration, the extended configuration, or the one or more intermediate positions.

### Example 64

The method of any one of the preceding examples, but particularly Example 63, wherein the control element comprises a material having shape memory and pseudo elastic properties.

### Example 65

The method of any one of the preceding examples, but particularly Example 64, wherein the control element comprises shape memory alloy.

### Example 66

The method of any one of the preceding examples, but particularly Example 65, wherein the linear actuator comprises a device configured to deliver current to the control element to elongate or shorten the control element.

### Example 67

The method of any one of the preceding examples, but particularly Example 60, wherein the blood vessel is a superior vena cava or an inferior vena cava.

### Example 68

The method of any one of the preceding examples, but particularly Example 60, wherein the implantable device further comprises: a sensor; and an actuation device comprising: a linear actuator coupled to a control element, a power source, and a microcontroller electrically coupled to the power source, the linear actuator, and the sensor, and wherein the method further comprises: detecting a blood pressure with the sensor; categorizing the detected blood pressure as within a predetermined first pressure state, a predetermined second pressure state, or a predetermined third pressure state that is between the first and second pressure states, wherein the categorizing is performed by the microcontroller; and adjusting the actuation device wherein adjusting the actuation device is one of: activating the linear actuator to tension the control element to maintain the coilable flow occlusion strip in the collapsed configuration in response to the first pressure state; activating the linear actuator to release tension in the control element to maintain the coilable flow occlusion strip in the extended configuration in response to the second pressure state; and activating the linear actuator to tension the control element to maintain the coilable flow occlusion strip in one or more intermediate positions in response to the third pressure state.

### Example 69

The method of any one of examples 60-68, further comprising restricting blood flow within the blood vessel.

### Example 70

The method of any one of examples 60-68, further comprising treating a subject having one or both of: congestive heart failure or chronic kidney disease.

### Example 71

An implantable device for modulating blood flow through a blood vessel, the implantable device comprising: a support frame; and an elongated membrane adapted to flex between a collapsed configuration and an extended configuration, wherein the elongated membrane has a first end coupled to the support frame and a second end that is configured to: stretch to cause the elongated membrane to form an elongated spiral with relative to the support frame, or contract to form a substantially flat coil relative to the support frame.

### Example 72

A method of restricting blood flow within a blood vessel, comprising using the device of example 71 to restrict blood flow within the blood vessel.

### Example 73

A method of treatment for a subject having one or both of: congestive heart failure or chronic kidney disease, comprising using the device of example 71 to restrict blood flow within the blood vessel.

### Example 74

An implantable device for modulating blood flow through a blood vessel, the implantable device comprising: an expandable cage having a first end opposite a second end; and one or more leaflets extending between the first end and the second end, wherein the expandable cage is movable between a collapsed configuration in which the one or more leaflets are configured to at least partially prolapse and an expanded configuration in which the one or more leaflets are configured to elongate or extend between the first and second ends.

### Example 75

The implantable device of any one of the preceding examples, but particularly Example 74, wherein the expandable cage comprises two or more wires connecting the first end to the second end.

### Example 76

The implantable device of any one of the preceding examples, but particularly Example 74, wherein the expandable cage comprises a plurality of wires connecting the first end to the second end.

### Example 77

The implantable device of any one of the preceding examples, but particularly Example 75, wherein the two or more wires have an arcuate shape in the expanded configuration and a partial spiral shape in the collapsed configuration.

### Example 78

The implantable device of any one of the preceding examples, but particularly Example 74, wherein the expandable cage comprises Nitinol.

### Example 79

The implantable device of any one of the preceding examples, but particularly Example 74, wherein the one or more leaflets in the collapsed configuration are configured to at least partially occlude the blood vessel in which the implantable device is disposed.

### Example 80

The implantable device of any one of the preceding examples, but particularly Example 74, further comprising one or more intermediate configurations between the collapsed configuration and the expanded configuration.

### Example 81

The implantable device of any one of the preceding examples, but particularly Example 74, wherein the blood vessel is an inferior vena cava or a superior vena cava.

### Example 82

The implantable device of any one of the preceding examples, but particularly Example 74, further comprising a lock on the first end or the second end, wherein the lock is configured to maintain the implantable device in the expanded configuration.

### Example 83

A method of restricting blood flow within a blood vessel, comprising using the device of any one of examples 74-82 to restrict blood flow within the blood vessel.

### Example 84

A method of treatment for a subject having one or both of: congestive heart failure or chronic kidney disease, comprising using the device of any one of examples 74-82 to restrict blood flow within the blood vessel.

### Example 85

An implantable device for modulating blood flow through a blood vessel, the implantable device comprising: a frame defining an inflow end and an outflow end; one or more paddles pivotally coupled to the inflow end or the outflow end; and a stop configured to translate relative to the one or more paddles to adjust a degree of rotation of the one or more paddles relative to a longitudinal axis of the frame, wherein the one or more paddles are movable between a closed configuration in which the one or more paddles are substantially perpendicular to the longitudinal axis of the frame and an open configuration in which the one or more paddles are substantially parallel to the longitudinal axis of the frame.

### Example 86

A method of treatment for a subject having one or both of: congestive heart failure or chronic kidney disease, comprising using the device of example 85 to restrict blood flow within the blood vessel.

What is claimed is:

1. An implantable device for modulating blood flow through a blood vessel, the implantable device comprising:
a support frame;
a coiled wire forming a coil stack, wherein the coiled wire is adapted to flex between a collapsed configuration and an extended configuration, wherein the coiled wire has a first end and a second end, and wherein the first end is coupled to or formed from the support frame and the second end is extendable and collapsible relative to the support frame; and
a jacket surrounding at least a portion of the coiled wire.

2. The device of claim 1, wherein the coiled wire is conical and wherein the formed coil stack is conical.

3. The device of claim 1, wherein the coiled wire comprises a cross-section that includes one of: a first moment of area wherein a bending resistance of the coiled wire differs between two or more orthogonal axes within a cross-sectional plane, or a second moment of area wherein a bending resistance of the coiled wire is substantially equal about a pair of orthogonal axes within the cross-sectional plane.

4. The device of claim 1, wherein the coiled wire is adapted to flex to one or more intermediate positions between the collapsed configuration and the extended configuration.

5. The device of claim 4, wherein, when the coiled wire is at least partially flexed, the second end is configured to extend away from the first end along a substantially longitudinal axis of the support frame in a same direction as a blood flow direction through the blood vessel.

6. The device of claim 4, wherein the coiled wire is adapted to flex to one or more of: the collapsed configuration, the extended configuration, or the one or more intermediate positions, in response to a blood pressure acting on the device.

7. The device of claim 1, wherein the coiled wire comprises a material with shape memory properties.

8. The device of claim 7, wherein the shape memory of the coiled wire is a shape of the coiled wire in the collapsed configuration.

9. The device of claim 1, wherein the jacket comprises a material, or a combination of materials, appropriate for creating a seal.

10. The device of claim 9, wherein, in the collapsed configuration, the jacket forms the seal between a first coil and a second coil.

11. The device of claim 10, wherein, in the collapsed configuration, the jacket comprises a cross-section shape complementary to itself when conically stacked.

12. An implantable device for modulating blood flow through a blood vessel, the implantable device comprising:
a support frame; and
a plurality of coiled wires forming a coil stack narrowing in a direction parallel to a longitudinal axis of the support frame and away from an end of the support frame, wherein each coiled wire is adapted to flex between a collapsed configuration and an extended configuration, wherein each coiled wire has a fixed end and a terminus, and wherein the fixed end of each coiled wire is coupled to or formed from the end of the support frame and the terminus of each coiled wire is extendable and collapsible relative to the support frame, and
wherein each coiled wire includes a respective jacket surrounding at least a portion of each coiled wire.

13. The device of claim 12, wherein, in the collapsed configuration, the respective jackets of the coiled wires are complementary to one another.

14. The device of claim 12, wherein the plurality of coiled wires is conical and wherein the formed coil stack is conical.

15. The device of claim 12, wherein the plurality of coiled wires is approximately parallel.

16. An implantable valve for enabling blood flow in a first blood flow direction and preventing blood flow in a second blood flow direction, the implantable valve comprising:
a support frame;
a coiled wire forming a coil stack, wherein the coiled wire is adapted to flex between a collapsed configuration and an extended configuration, wherein the coiled wire has a first end and a second end, and wherein the first end is coupled to or formed from an end of the support frame and the second end is extendable and collapsible relative to the support frame, and wherein the coil stack extends longitudinally outward beyond the end of the support frame; and
a jacket surrounding at least a portion of the coiled wire.

17. The implantable valve of claim 16, wherein the coiled wire is conical and wherein the formed coil stack is conical.

18. The implantable valve of claim 16, wherein the coiled wire comprises a cross-section that includes one of: a first moment of area wherein a bending resistance of the coiled wire differs between two or more orthogonal axes within a cross-sectional plane, or a second moment of area wherein a bending resistance of the coiled wire is substantially equal about a pair of orthogonal axes within the cross-sectional plane.

19. The implantable valve of claim 16, wherein the coiled wire is adapted to flex to one or more intermediate positions between the collapsed configuration and the extended configuration.

20. The implantable valve of claim 19, wherein, when the coiled wire is at least partially flexed, the second end is configured to extend away from the first end along a substantially longitudinal axis of the support frame in the first blood flow direction.
